(19) United States
(12) Patent Application Publication
(10) Pub. No.: US 2026/0162831 A1
(43) Pub. Date: Jun. 11, 2026

(54) DIGITAL TWIN SYSTEM FOR PULMONARY HEALTHCARE

(71) Applicant: The Board of Regents for the Oklahoma Agricultural and Mechanical colleges, Stillwater, OK (US)

(72) Inventors: Yu Feng, Stillwater, OK (US); Jianan Zhao, Silver Spring, MD (US)

(21) Appl. No.: 19/179,837

(22) Filed: Apr. 15, 2025

Related U.S. Application Data
(63) Continuation of application No. PCT/US23/76979, filed on Oct. 16, 2023.
(60) Provisional application No. 63/380,160, filed on Oct. 19, 2022.

(51) Int. Cl. G16H 50/50; G06F 30/28

(52) U.S. Cl. CPC G16H 50/50; G06F 30/28

(57) ABSTRACT

Systems and methods for providing a virtual whole-lung model of a patient respiratory system are herein disclosed, including a non-transitory computer readable medium storing a set of computer readable instructions that when executed by a processor cause the processor to: determine a model of airway deformation in a patient respiratory system using an elastic truncated whole-lung (TWL) model, the model of airway deformation having at least one designated lung site; determine a plurality of particle airflows in the patient respiratory system for at least one disease specific level; and determine drug delivery efficiency to the designated lung site using the model of airway deformation and the plurality of particle airflows in the patient respiratory system.

### CROSS-REFERENCE TO RELATED APPLICATIONS

This application claims priority to the international patent application identified by PCT/US2023/076979, filed on Oct. 16, 2023, which claims priority to provisional patent application identified by U.S. Ser. No. 63/380,160, filed Oct. 19, 2022, the entire content of both of the international patent application and the provisional patent application are hereby expressly incorporated herein by reference.

### FEDERALLY SPONSORED RESEARCH AND DEVELOPMENT

This invention was made with government support under Grant CBET-2120688 awarded by the National Science Foundation. The government has certain rights in the invention.

### BACKGROUND ART

The impact of chronic lung diseases, such as asthma and chronic obstructive pulmonary disease (COPD), is a globally growing concern. Treatment of these ailments may include a variety of interventions, including orally inhaled drug products (OIDPs), such as dry powder inhalers (DPIs).

Spiriva™ Handihaler™ is one example of a DPI that delivers an efficacious dose of active pharmaceutical ingredient (API) nanoparticles to designated lung sites, e.g., peripheral lung, to treat emphysema as one of the three contributors to COPD. Upon actuation via patient inhalation, a dry powder dosage under the influence of inspiratory airflow is entrained and deagglomerated by a variety of fluidization and dispersion mechanisms that are device-specific. In addition, dry powders may also contain micron-sized carrier particles (e.g., lactose carrier particles) to increase API particle dispersion, thereby improving the delivery efficiency of APIs to the peripheral lung.

In 2017, the US Food and Drug Administration (FDA) published the Generic Drug User Fee Amendments (GDUFA) to enable reviewers to assess abbreviated new drug applications (ANDAs) more efficiently with an emphasis on regulatory science enhancements of complex drug products, including OIDPs. For some orally administered drugs that reach sites of action through systemic circulation, bioequivalence is demonstrated based on drug concentration in a relevant biologic fluid (e.g., plasma or blood). However, this approach is currently considered inadequate in the United States to establish bioequivalence of inhalation products intended for local action, as the lung delivery does not rely on the systemic circulation. Instead, the comparability between generic DPIs and the reference listed drug (RLD) DPIs is based on (1) device delivery efficiency, (2) emitted aerodynamic particle size distributions (APSDs), (3) lung deposition, and (4) equivalent pharmacokinetics (PK) and clinical/pharmacodynamics (PD) data, with the latter being an indicator of local delivery.

### SUMMARY OF THE INVENTION

Effective inhalation therapy using DPIs depends on the total mass of the API from the DPI mouthpieces and the APSDs. Thus, accurate predictions of emitted APSDs from DPIs and the resultant lung deposition of OIDPs is a first step to demonstrating the comparability between different designs of DPIs. However, achieving comparability in emitted APSDs and lung depositions may be challenging because such comparability is related to DPI performance, which is a function of interactions between the patient and device (i.e., breathing patterns), as well as drug particle characteristics. Specifically, the deagglomeration and agglomeration between APIs and carrier particles require a detailed understanding, since deagglomeration and agglomeration are the key mechanisms influencing the emitted APSD. Therefore, new insights into DPI product developments are critically needed, which requires support from high-resolution particle dynamics data provided by reliable numerical models in a cost-effective and time-saving manner. There is a need to develop a reliable computational model to provide high-resolution in silico supportive evidence on air-particle flow dynamics both in the DPI flow channel and in virtual human respiratory systems, which can predict particle transport and entrainment with particle-particle interactions, including agglomeration/deagglomeration.

In one aspect, the present disclosure relates to a non-transitory computer readable medium storing a set of computer readable instructions that when executed by a processor cause the processor to: determine a model of airway deformation in a physiologically realistic patient-specific respiratory environment using an elastic truncated whole-lung (TWL) model, the model of airway deformation having at least one designated lung site; determine a plurality of particle airflows in the patient respiratory system for at least one disease specific level; and, determine drug delivery efficiency to the designated lung site using the model of airway deformation and the plurality of particle airflows in the patient respiratory system.

In another aspect, the present disclosure relates to a non-transitory computer readable medium storing a set of computer readable instructions that when executed by a processor cause the processor to: generate a one-way coupled Computational Fluid Dynamics (CFD) with Discrete Element Method (DEM) virtual whole-lung model of a patient respiratory system using Hertz-Mindlin (H-M) Johnson-Kendall-Roberts (JKR) cohesion model (CFD-DEM virtual whole-lung model), the CFD-DEM virtual whole-lung model configured to predict particle agglomeration and deagglomeration with resultant emitted APSDs; calibrate the CFD-DEM virtual whole-lung model; validate the CFD-DEM virtual whole-lung model; and, determine drug delivery efficiency and drug delivery deposition patterns of a DPI within the patient respiratory system using the CFD-DEM virtual whole-lung model.

In another aspect, the present disclosure relates to a method, comprising: generating, by one or more processor, a one-way coupled CFD-DEM virtual whole-lung model configured to predict particle agglomeration and deagglomeration with resultant emitted APSDs; calibrating, by the one or more processor, the CFD-DEM virtual whole-lung model; validating, by the one or more processor, the CFD-DEM virtual whole-lung model; and, determining, by the one or more processor, drug delivery efficiency and drug delivery deposition patterns of a DPI within the patient respiratory system using the CFD-DEM virtual whole-lung model.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIGS. 1A and 1B are composite views of exemplary embodiments of an inhaler for use in accordance with the present disclosure;

FIG. 2 is a diagrammatic view of an exemplary embodiment of a digital twin system constructed in accordance with the present disclosure;

FIG. 3A is a diagrammatic view of a geometry and a polyhedral mesh with a near-wall prism layer of a patient respiratory system constructed in accordance with the present disclosure;

FIG. 3B is a diagrammatic view of a particle-particle interaction in an H-M model with JKR cohesion for a DEM in accordance with the present disclosure;

FIG. 4 is a graphical view of a relationship between JKR particle-wall surface energy and DPI delivery efficiency predicted by an in situ model in accordance with the present disclosure;

FIGS. 5A and 5B are diagrammatic views of airflow structures within a flow channel using an in situ model of a first inhaler shown in FIG. 1A;

FIG. 6 is a diagrammatic view of particle deposition in the flow channel shown in FIGS. 5A and 5B;

FIGS. 7A and 7B are graphical views of particle deposition in the flow channel and delivery efficiency of the first inhaler shown in FIG. 1A;

FIGS. 8A-8D are graphical views of effects of particle shape and actuation flow rate (Qᵢₙ) on emitted APSD using the first inhaler shown in FIG. 1A;

FIGS. 9A-9C are graphical views of an effect of Qᵢₙ on emitted APSD for the first inhaler shown in FIG. 1A;

FIGS. 10A and 10B are diagrammatic views of inspiratory airflow structures at the sagittal plane y=0 for the three-dimensional patient respiratory system shown in FIG. 3A;

FIG. 11A is a diagrammatic view of lactose delivery deposition patterns in an upper airway at different Qsᵢₙ using the first inhaler shown in FIG. 1A;

FIG. 11B is a graphical view of the lactose delivery deposition patterns in the upper airway shown in FIG. 11A at different Qsᵢₙ;

FIG. 12A is a diagrammatic view of lung deposition patterns of APIs and regional deposition fractions (RDF_{API-lung}) with different Qsᵢₙ and lactose aspect ratios (ARs), respectively, for the first inhaler shown in FIG. 1A;

FIG. 12B is a graphical view of lung deposition patterns of APIs and RDFs_{API-lung} with different Qsᵢₙ and lactose ARs, respectively, for the first inhaler shown in FIG. 1A;

FIGS. 13A and 13B are diagrammatic views of airflow structures within a flow channel using an in situ model of a second inhaler shown in FIG. 1B;

FIG. 14 is a diagrammatic view of particle delivery deposition in the flow channel shown in FIGS. 13A and 13B and delivery efficiency of the second inhaler shown in FIG. 1B;

FIGS. 15A and 15B are graphical views of particle delivery deposition in the flow channel shown in FIGS. 13A and 13B and delivery efficiency of the first inhaler shown in FIG. 1A and the second inhaler shown in FIG. 1B;

FIG. 16 is a graphical view of an effect of Qᵢₙ on emitted APSD for the second inhaler shown in FIG. 1B;

FIG. 17A is a diagrammatic view of lactose delivery deposition patterns in an upper airway at different Qsᵢₙ using the second inhaler shown in FIG. 1B;

FIG. 17B is a graphical view of lactose delivery deposition patterns in the upper airway shown in FIG. 17A at different Qsᵢₙ;

FIG. 18A is a diagrammatic view of lung deposition patterns of APIs and RDFs_{API-lung} with different Qsᵢₙ and lactose ARs, respectively, for the second inhaler shown in FIG. 1B;

FIG. 18B is a graphical view of lung deposition patterns of APIs and RDFs_{API-lung} with different Qsᵢₙ and lactose ARs, respectively, for the second inhaler shown in FIG. 1B;

FIGS. 19 and 20A-20F are diagrammatic views of another exemplary embodiment of an in situ model configured to reconstruct an airways tree such that airways branch follows the rules of regular dichotomy after generation 3 (G3) to generation 17 (G17) constructed in accordance with the present disclosure;

FIGS. 21A and 21B are diagrammatic views of deformation kinematics of a tracheobronchial (TB) tree in accordance with the present disclosure;

FIG. 22 is a graphical view of validation of the in situ model shown in FIGS. 19 and 20A-20F;

FIGS. 23A-23C are graphical views of a calibration of lung volume change predictions using the in-situ model of FIGS. 19 and 20A-20F via matching pulmonary function test (PFT) data for different lung disease conditions;

FIGS. 24A-24F are diagrammatic views of normalized velocity magnitude contours at a sagittal plane in accordance with the present disclosure;

FIGS. 25A-25F are diagrammatic views of normalized velocity magnitude contours and tangential velocity vectors on selected slices at

$$t = \frac{1}{4} {T}_{c}$$

in accordance with the present disclosure;

FIG. 26A-26F are diagrammatic views of lung deposition patterns of particles with multiple diameters in accordance with the present disclosure;

FIG. 27 is a graphical view of total deposition fractions (DFs) of particles in a whole lung model with different diameters under different lung health conditions in accordance with the present disclosure;

FIGS. 28A-28G are graphical views of comparisons of regional DF (RDF) predictions via a static truncated whole lung (TWL) model and an elastic TWL model under three lung health conditions for particles with different diameters in accordance with the present disclosure; and

FIGS. 29A-29C are graphical views of comparisons of RDFs predicted via the elastic TWL model under different lung disease conditions in accordance with the present disclosure.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), field programmable gate array (FPGA), a combination of hardware and software, and/or the like. The term “processor” as used herein means a single processor or multiple processors working independently or together to collectively perform a task.

Software may include one or more computer readable instructions that when executed by one or more components cause the component to perform a specified function. It should be understood that the algorithms described herein may be stored on one or more non-transitory memory. Exemplary non-transitory memory may include random access memory, read only memory, flash memory, and/or the like. Such non-transitory memory may be electrically based, optically based, and/or the like.

The term “patient” as used herein includes human and veterinary subjects.

Certain exemplary embodiments of the invention will now be described with reference to the drawings. In some embodiments, an in silico model is configured to provide a benchmark pathway to utilize in vitro and in vivo clinical data to provide disease-specific diagnosis and/or treatment. In some embodiments, the in silico model may provide determination of carrier-API interactions in dry powder inhalers (DPIs), effect of lactose carrier shape (i.e., the shape of lactose carrier particles) on drug delivery efficiency, and DPI flow channel design (i.e., dry powder inhaler flow channel design) on drug delivery efficiency and/or drug delivery deposition pattern(s) in a patient respiratory system. The in silico model may be a virtual whole-lung model that encompasses the entire pulmonary route from mouth and/or nose to alveoli. In some embodiments, the in silico model may be configured to evaluate lung uptakes of inhaled aerosolized medications. In some embodiments, the in silico model may be used to determine optimized design of an inhaler, inhaled drug design, and/or the like.

In general, embodiments describe herein may relate to systems and methods for computer-assisted computational fluid dynamics-discrete element method (CFD-DEM) and computational fluid-particle dynamics (CFPD) providing relationships between DPI design, lactose carrier particle shape, Qᵢₙ between patient and DPI, and/or the drug delivery efficiency to specific pre-determined lung regions. In some embodiments, such systems and methods may determine fundamental carrier-API interactions in DPIs, effect of lactose carrier particle shape and/or DPI flow channel designs on drug delivery efficiency from DPI, and/or drug delivery deposition patterns within a patient respiratory system.

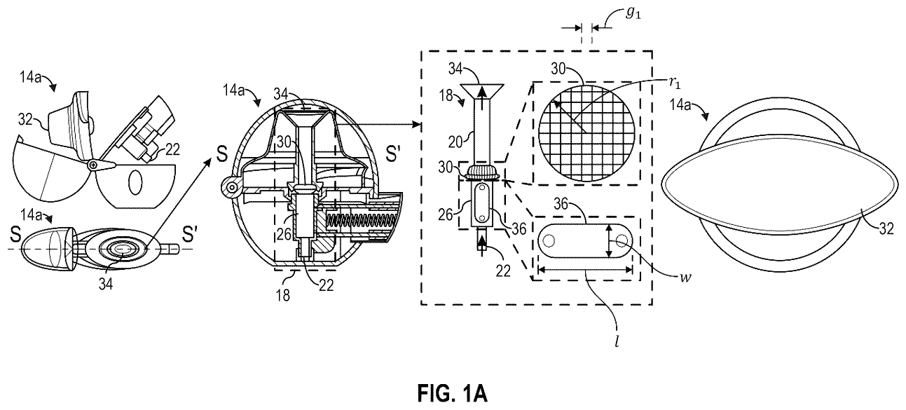

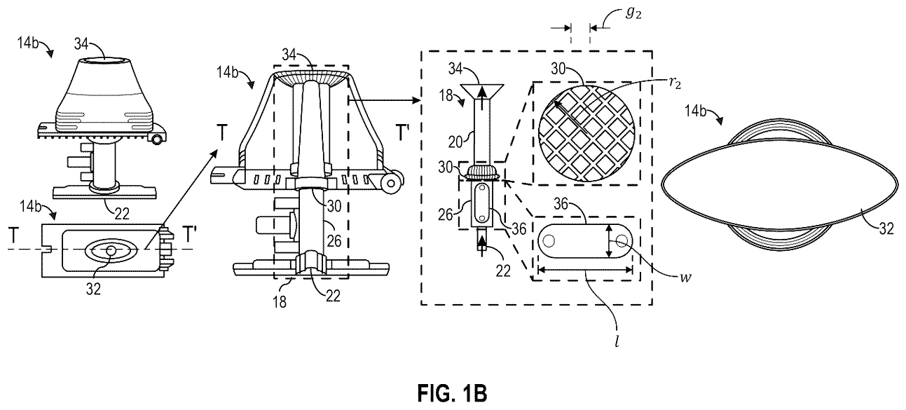

Turning now to the drawings, and in particular to FIGS. 1A and 1B, shown therein are exemplary embodiments of a first inhaler 14a and a second inhaler 14b (either of the first inhaler 14a and the second inhaler 14b, hereinafter the “inhaler 14”, and collectively the “inhalers 14”) constructed in accordance with the present disclosure. The inhaler 14 may be configured to deliver an efficacious dose of API nanoparticles to designated lung sites (e.g., peripheral lung). Upon actuation via patient inhalation, the inhaler 14 may be configured to provide a dry powder dosage, for example, under the influence of inspiratory airflow.

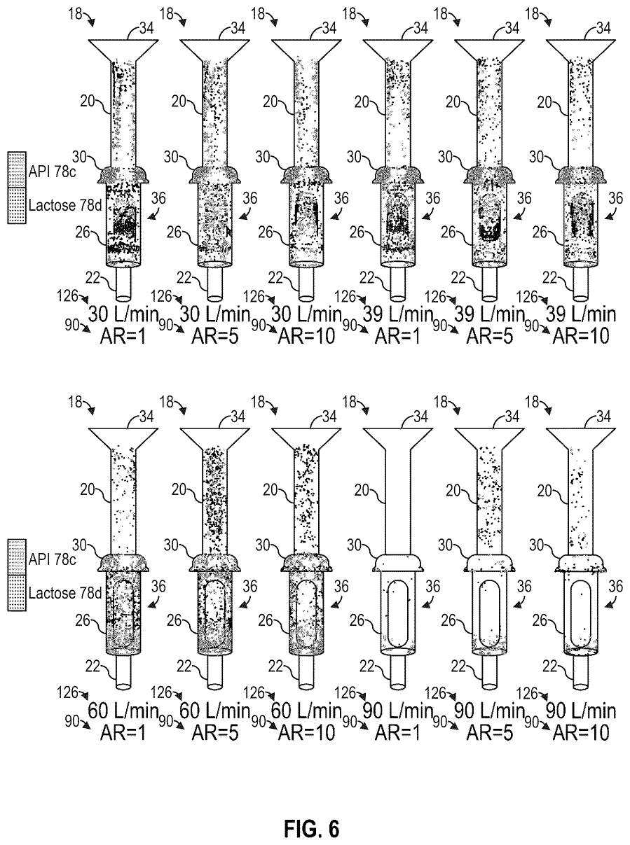

In some embodiments, the inhaler 14 may be a dry powder inhaler (DPI). In such embodiments, the first inhaler 14a may be a Spiriva™ Handihaler™ DPI, and the second inhaler 14b may be an alternative DPI. The dry powder dosage may be entrained and deagglomerated by a variety of fluidization and dispersion mechanisms that may be device-specific. In addition, dry powders may contain micron-sized carrier particles (e.g., lactose carrier particles 78d (shown in FIG. 6)) to increase dispersion of API particles 78c (shown in FIG. 6), thereby improving the delivery efficiency of API particles 78c to the peripheral lung.

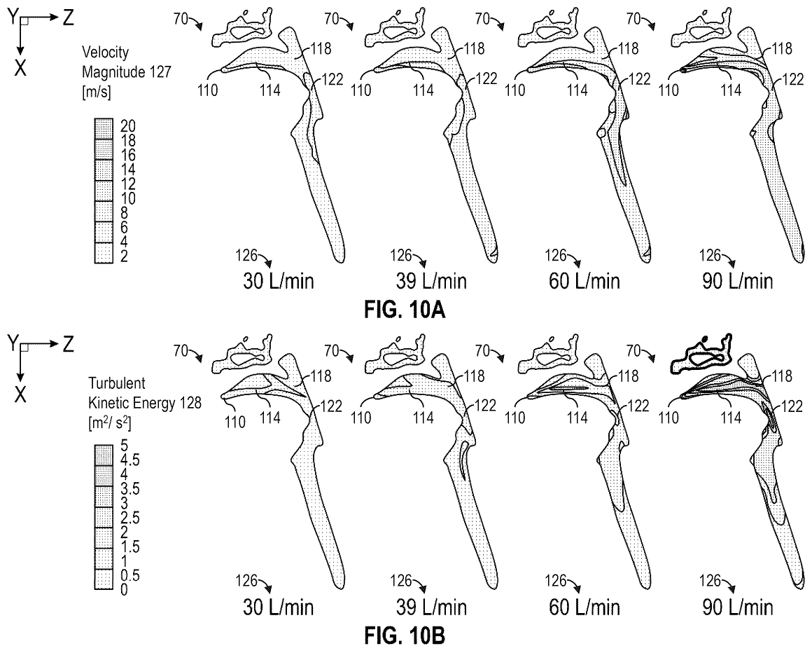

The inhaler 14 may include at least one flow channel 18 (hereinafter the “flow channel 18”) as illustrated in FIGS. 1A and 1B. In some embodiments, the flow channel 18 is defined by an inner wall 20 (hereinafter “the wall 20”). The flow channel 18 may contain an elliptical actuation air inlet 22. Additionally, the flow channel 18 may contain at least one capsule chamber 26 (hereinafter the “capsule chamber 26”). In some embodiments, the capsule chamber 26 may have a diameter of 7.5 mm and a length of 17.8 mm along the flow direction for at least one inhaler 14. As shown in FIGS. 1A and 1B, one or more grid 30 (hereinafter the “grid 30”) may be included to separate particle bulk flows. The flow channel 18 may also include one or more extended tube and/or elliptic mouthpiece 34 (hereinafter the “mouthpiece 34”) as outlets connecting to the oral cavity 114 (shown in FIG. 10A). One or more capsule 36 (hereinafter the “capsule 36”) may be positioned at a center of the capsule chamber 26.

As shown in FIG. 1A, the grid 30 of the first inhaler 14a may have a radius r₁ of 5 mm and a grid spacing g₁ of 1 mm. As shown in FIG. 1B, the grid 30 of the second inhaler 14b may have a radius r₂ of 4.5 mm and a grid spacing g₂ of 1.2 mm. Further, the capsule 36 in either of the inhalers 14 may have a length l of 15 mm and a width w of 5 mm.

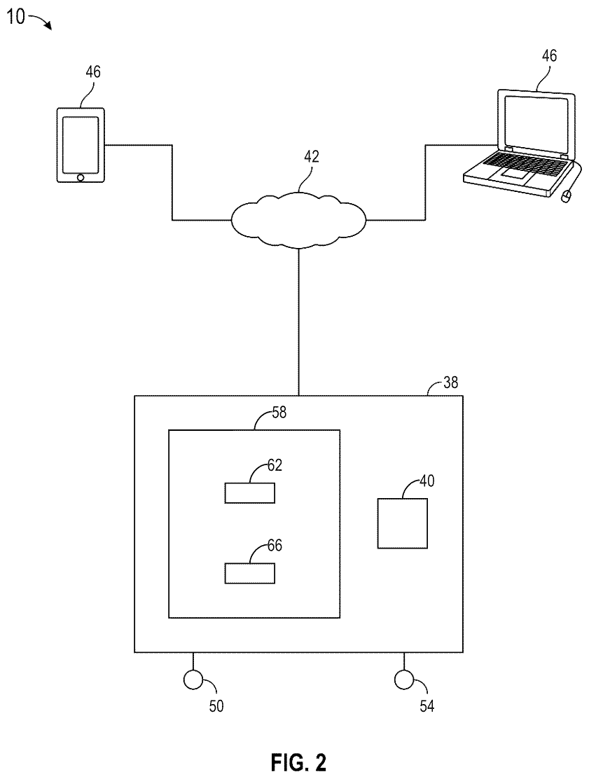

Referring to FIG. 2, the system 10 may be a system or systems that are able to embody and/or execute the logic of the processes described herein. Logic embodied in the form of software instructions and/or firmware may be executed on any appropriate hardware. For example, logic embodied in the form of software instructions or firmware may be executed on a system or systems, or on a personal computer system, or on a distributed processing computer system, and/or the like. In some embodiments, logic may be implemented in a stand-alone environment operating on a single computer system and/or logic may be implemented in a networked environment, such as a distributed system using multiple computers and/or processors networked together.

In some embodiments, the system 10 may include one or more computer system 38 (hereinafter the “computer system 38”) comprising one or more processor 40 (hereinafter the “processor 40”). The processor 40 may work to execute processor executable code. The processor 40 may be implemented as a single or plurality of processors working together, or independently, to execute the logic as described herein. Exemplary embodiments of the processor 40 may include, but are not limited to, a digital signal processor (DSP), a central processing unit (CPU), a field programmable gate array (FPGA), a microprocessor, a multi-core processor, and/or combinations thereof, for example. In some embodiments, the processor 40 may be incorporated into a smart device.

It is to be understood that in certain embodiments using more than one processor, the processors 40 may be located remotely from one another, in the same location, or comprising a unitary multi-core processor. In some embodiments, the processor 40 may be partially or completely network-based or cloud-based, and may or may not be located in a single physical location. The processor 40 may be capable of reading and/or executing processor-executable code and/or capable of creating, manipulating, retrieving, altering, and/or storing data structure into one or more memories.

The processor 40 may be capable of communicating via a network 42 or a separate network (e.g., analog, digital, optical, and/or the like). In some embodiments, the processor 40 may transmit and/or receive data via the network 42 to and/or from one or more external systems 46 (hereinafter the “external systems 46”) (e.g., one or more external computer systems, one or more machine learning applications, artificial intelligence, cloud-based system, microphones). For example, the processor 40 may allow users (e.g., healthcare providers, physicians, medical personnel) of the external systems 46 access via the network 42 to provide and/or receive data. Access methods include, but are not limited to, cloud access and direct download to the processor 40 via the network 42. In some embodiments, the processor 40 may be provided on a cloud cluster (i.e., a group of nodes hosted on virtual machines and connected within a virtual private cloud). Additionally, processors 40 may provide data to a user by methods that include, but are not limited to, messages sent through the processor 40 and/or external systems 46, SMS, email, and telephone. It is to be understood that in some exemplary embodiments, the processor 40 and the one or more external systems 46 may be implemented as a single device.

The one or more external systems 46 may be configured to provide information and/or data in a form perceivable to the processor 40. For example, the one or more external systems 46 may include, but are not limited to, implementations as a laptop computer, a computer monitor, a screen, a touchscreen, a microphone, a website, a smart phone, a PDA, a cell phone, an optical head-mounted display, combinations thereof, and/or the like. The external systems 46 may provide data in computer readable form, such as a text file, a word document, and/or the like.

As used herein, the terms “network-based”, “cloud-based”, and any variations thereof, may include the provision of configurable computational resources on demand via interfacing with a computer and/or computer network, with software and/or data at least partially located on a computer and/or computer network, by pooling processing power of two or more networked processors.

In some embodiments, the network 42 may be the Internet and/or other network. For example, if the network 42 is the Internet, a primary user interface of the medical coding software may be delivered through a series of web pages. It should be noted that the primary user interface of the medical billing software may be via any type of interface, such as, for example, a Windows-based application.

The network 42 may be almost any type of network. For example, the network 42 may interface via optical and/or electronic interfaces, and/or may use a plurality of network topographies and/or protocols including, but not limited to, Ethernet, TCP/IP, circuit switched paths, combinations thereof, and the like. For example, in some embodiments, the network 42 may be implemented as the World Wide Web (or Internet), a local area network (LAN), a wide area network (WAN), a metropolitan network, a wireless network, a cellular network, a Global System of Mobile Communications (GSM) network, a code division multiple access (CDMA) network, a 4G network, a 5G network, a satellite network, a radio network, an optical network, an Ethernet network, combinations thereof, and/or the like. Additionally, the network 42 may use a variety of network protocols to permit bi-directional interface and/or communication of data and/or information. It is conceivable that in the near future, embodiments of the present disclosure may use more advanced networking topologies.

In some embodiments, the system 10 may include one or more input device 50 (hereinafter the “input device 50”) and one or more output device 54 (hereinafter the “output device 54”). The input device 50 may be capable of receiving information from a user, processors, and/or environment, and transmit such information to the processor 40 and/or the network 42. The input device 50 may include, but is not limited to, implementation as a keyboard, touchscreen, mouse, trackball, microphone, fingerprint reader, infrared port, slide-out keyboard, flip-out keyboard, cell phone, PDA, video game controller, remote control, network interface, speech recognition, gesture recognition, combinations thereof, and/or the like.

The output device 54 may be capable of outputting information in a form perceivable by a user, the external systems 46, and/or the processor 40. For example, the output device 54 may include, but is not limited to, implementation as a computer monitor, a screen, a touchscreen, a speaker, a website, a television set, a smart phone, a PDA, a cell phone, a fax machine, a printer, a laptop computer, an optical head-mounted display (OHMD), combinations thereof, and/or the like. It is to be understood that in some exemplary embodiments, the input device 50 and the output device 54 may be implemented as a single device, such as, for example, a touchscreen or a tablet.

The processor 40 may be capable of reading and/or executing processor-executable code and/or capable of creating, manipulating, retrieving, altering and/or storing data structures into one or more non-transitory computer readable medium 58 (hereinafter the “memory 58”). The processor 40 may include one or more non-transient computer readable medium comprising processor-executable code and/or one or more software application. In some embodiments, the memory 58 may be located in the same physical location as the processor 40. Alternatively, one or more memory 58 may be located in a different physical location as the processor 40 and communicate with the processor 40 via a network (e.g., the network 42). Additionally, one or more memory 58 may be implemented as a “cloud memory” (i.e., one or more memory may be partially or completely based on or accessed using a network (e.g., the network 42).

The memory 58 may store processor-executable code and/or information comprising one or more database 62 (hereinafter the “database 62”) and program logic 66 (i.e., computer executable logic). In some embodiments, the processor-executable code may be stored as a data structure, such as a database and/or data table, for example. In some embodiments, one or more database 62 may store one or more predefined dictionaries via the methods described herein. In use, the processor 40 may execute the program logic 66 controlling the reading, manipulation, and/or storing of data as detailed in the processes described herein.

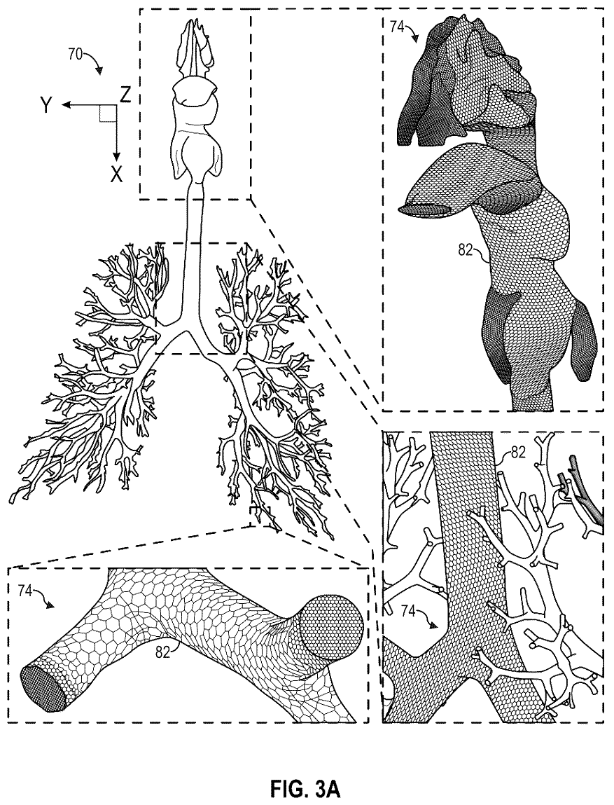

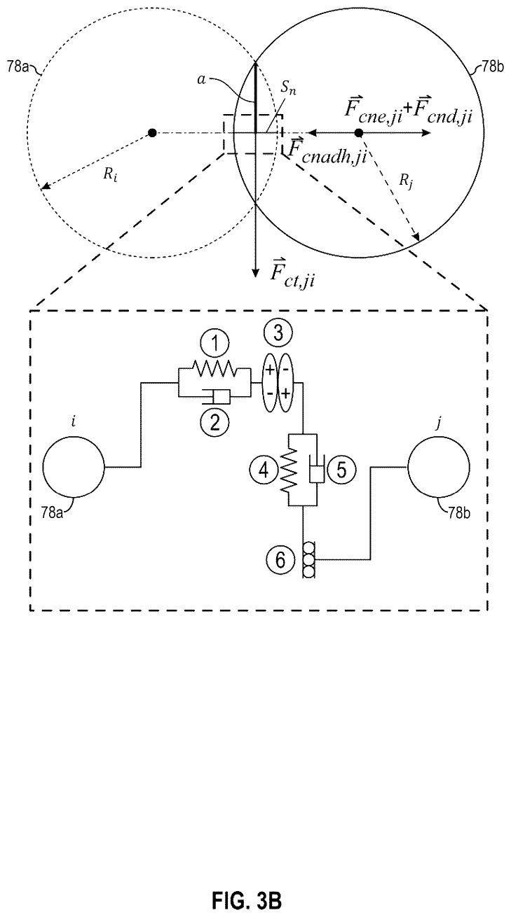

In some embodiments, the inhaler 14 may be computationally modeled using the processor 40. For example, the inhaler 14 may be computationally modeled to include the flow channel 18 as illustrated in FIGS. 1A and 1B. In some embodiments, finite volume meshes may be used for the flow channel 18. Meshes may consist of polyhedral elements with near-wall prism layers configured to capture the laminar-to-turbulence transitions accurately using the Generalized k-ω (GEKO) turbulence model. Meshes of the inhaler 14 may include a total between 3,732,269-2,936,375 cells, for example. In some embodiments, 7,064,092 polyhedron-based cells may be generated for the computational domain of a patient respiratory system 70 (shown in FIG. 3A). In some embodiments, near-wall prism layers may be generated (e.g., five near-wall prism layers), to resolve the velocity gradient and precisely capture the laminar-to-turbulence transitions close to the wall 20, for example.

Referring now to FIG. 3A, shown therein is a three-dimensional (3D) human respiratory system geometry 70 (hereinafter the “patient respiratory system 70”) which may be constructed by extending mouth/nose-to-trachea geometry used in the prior art with a 3D tracheobronchial tree covering up to generation 13 (G13). An overview of the patient respiratory system 70 and a CFD mesh 74 (hereinafter the “mesh 74”) is shown in FIG. 3A.

Accurate prediction of aerodynamic particle size distributions (APSDs) emitted from the inhaler 14 using the in situ model includes consideration of effects of particle-particle and particle-wall interactions (i.e., agglomeration and deagglomeration) during API particle transport simulations. To address such a complexity, a generalized one-way coupled CFD-DEM model with an H-M JKR cohesion model is calibrated and validated. As described in further detail herein, the validated CFD-DEM model may predict the particle agglomeration/deagglomeration and the resultant emitted APSDs (i.e., the resultant emitted aerodynamic particle size distributions) in a computationally efficient manner. Further, the H-M JKR model can accurately describe the adhesion resulting from the short-range surface force(s) for studies of agglomeration at micro-/nano-scale.

For air-particle flow dynamics simulations in the patient-specific respiratory system 70, the validated CFD-DEM model may be used. Specifically, turbulent airflow may be simulated using Reynolds-averaged Navier-Stokes (RANS) equations. For particle tracking, individual particle trajectories may be determined using a Lagrange method. Specifically, the particle trajectory and velocity may be determined by evaluation of forces acting on the particles (e.g., drag force, gravitational force, Brownian motion-induced force).

In some embodiments, within the validated CFD-DEM model, airflow in the inhalers 14 and patient respiratory systems 70 may be treated as a continuous phase. In contrast, particles embedded in the airflow may be considered discrete phases and tracked using the Lagrange method with the particle-particle interactions modeled using DEM. Conservation laws of mass and momentum for the airflow can be given as:

$$∇ \cdot {\vec{u}}_{f} = 0 \qquad EQ . ( 1 )$$

$$\frac{\partial {\vec{u}}_{f}}{\partial t} + ( {\vec{u}}_{f} \cdot ∇ ) {\vec{u}}_{f} = - \frac{∇ p}{{ρ}_{f}} + \frac{1}{{ρ}_{f}} ∇ \cdot ( \overline{\overline{{τ}_{f}}} ) + \vec{g} \qquad EQ . ( 2 )$$

Where [symbol] is the airflow velocity, and is the local stress tensor, calculated by:

$$\overline{\overline{{τ}_{f}}} = μ ( ∇ {\vec{u}}_{f} + ∇ {\vec{u}}_{f}^{T} ) \qquad EQ . ( 3 )$$

where μ is the molecular viscosity.

Translations, rotations, and interactions of API particles 78c and lactose carrier particles 78d (hereinafter “lactose particles 78d”) may be determined. A particle-particle interaction between a first particle 78a (i.e., particle i) and a second particle 78b (i.e., particle j) (collectively, the “particles 78”, and individually, each a “particle 78”), as well as force and torque balances for the second particle 78b, are shown in FIG. 3B. As shown in FIG. 3B, a is the contact radius and Sₙ is the normal overlap.

Governing equations for the discrete phase (i.e., the second particle 78b) may be given as:

$${m}_{p , j} \frac{d {\vec{u}}_{p , j}}{dt} = {\sum }_{i} {\vec{F}}_{c , ji} + {\vec{F}}_{fp , j} + {\vec{F}}_{g , j} \qquad EQ . ( 4 )$$

$${\vec{\vec{I}}}_{j} \frac{d {\vec{ω}}_{p , j}}{dt} = [ \begin{matrix}{I}_{xx} & {I}_{xy} & {I}_{xz} \\ {I}_{yx} & {I}_{yy} & {I}_{yz} \\ {I}_{zx} & {I}_{zy} & {I}_{zz}\end{matrix} ] [ \begin{matrix}\frac{{( d ω )}_{p , j , x}}{dt} \\ \frac{d {ω}_{p , j , y}}{dt} \\ \frac{d {ω}_{p , j , z}}{dt}\end{matrix} ] = {\sum }_{i} {\vec{M}}_{c , ji} + {\vec{M}}_{fp , j} \qquad EQ . ( 5 )$$

wherein m_{p,j} is the particle mass, [symbol] _{g,j} is the gravitational force, [symbol] _{c,ji} is particle-particle or particle-wall contact force, [symbol] _{fp,j} is the total force acting on the second particle 78b due to the fluid-particle interactions, [symbol] is the moment of inertia second-rank tensor, [symbol] _{p,j} is the angular velocity vector, [symbol] _{c,ji} is the contact torque induced by the tangential contact forces, and [symbol] _{fp,j} is the torque due to the airflow velocity gradient.

In EQ. 4, [symbol] _{fp} accounts for forces generated by the fluid on the particles 78, such as drag force [symbol] _{D}, the pressure gradient force [symbol] _{∇p}, added (virtual) mass force [symbol] _{VM}, lift force [symbol] _{L}, the Brownian motion induced force [symbol] _{BM}, and can be calculated using the Lagrange method by solving Newton's second law for each of the particles 78, i.e.:

$${\vec{F}}_{f p} = {\vec{F}}_{D} + {\vec{F}}_{∇ p} + {\vec{F}}_{V M} + {\vec{F}}_{L} + {\vec{F}}_{B M} ) \qquad ( EQ . 6 )$$

The majority of the forces in EQ. 6 may be ignored. Specifically, since the density difference between fluid and the particles 78 may be high (ρₚ>>ρ_{f}), [symbol] _{VM} and [symbol] _{L} can be neglected. In addition, since a size of the particles 78 is much smaller than a cell size of the mesh 74, [symbol] _{∇p} is negligible. Specifically, an edge length of the mesh 74 may be about 1 mm, whereas the median diameter of the lactose particles 78d and the API particles 78c may be 46 μm and 2.8 μm, respectively. The details of drag coefficients (C_{D}) selections for the particles 78 with both spherical and elongated sphero-cylindrical shapes are presented in the Supplemental Information (SI).

To model the deagglomeration and agglomeration behaviors among the API particles 78c and the lactose particles 78d with different diameters from 1 to 200 μm, the dominant adhesive forces (i.e., Van der Waals force and electrostatic force) may be integrated into the DEM contact force model. For example, the H-M model with JKR Cohesion may account for the adhesive behaviors between fine particles (i.e., the particles 78) and introduce a cutoff value for the inter-particulate distance to avoid the numerical singularity at particle contact. Specifically, the adhesive contact force may be modeled based on the balance between the stored elastic energy (i.e., normal and tangential elastic forces) and the loss in the surface energy (i.e., adhesion force). The H-M model with JKR cohesion describes particle contacts as normally and tangentially damped harmonic oscillators with tangential friction [symbol] _{cn,ji} and an adhesion force [symbol] _{ct,ji}. The JKR model includes the effect of elastic deformation, treats the effect of adhesion as surface energy only, and neglects adhesive stresses in the separation zone. Accordingly, inter-particle forces acting on the second particle 78b from the first particle 78a may be modeled by the summation of two forces in normal and tangential directions, i.e.:

$${\vec{F}}_{c , j i} = {\vec{F}}_{c n , j i} + {\vec{F}}_{ct , ji} \qquad ( EQ . 7 )$$

wherein [symbol] _{cn,ji} and [symbol] _{ct,ji} are normal and tangential contact forces, which can be expressed as:

$${\vec{F}}_{c n , j i} = {\vec{F}}_{c n e , j i} + {\vec{F}}_{c n d , j i} + {\vec{F}}_{c n a d h , j i} \qquad ( EQ . 8 )$$

$${\vec{F}}_{ct , ji} = {\vec{F}}_{cte , j i} + {\vec{F}}_{ctd , j i} + {\vec{F}}_{ctf , j i} \qquad ( EQ . 9 )$$

In EQ. 8, [symbol] _{cne,ji} is the normal elastic force, [symbol] _{cnd,ji} is the normal viscous damping force, and [symbol] _{cnadh,ji} is the adhesion force (i.e., adhesion resulting from short-range surface force of agglomeration) in the JKR cohesion model. Specifically, using the Hertz spring-dashpot model with JKR cohesion, the above-mentioned forces may be defined by:

$${\vec{F}}_{c n e , j i} = ( {K}_{n} {s}_{n}^{\frac{3}{2}} ) {\vec{n}}_{i j} = ( \frac{4}{3} {E}^{*} \sqrt{{R}^{⋆}} {s}_{n}^{\frac{3}{2}} ) {\vec{n}}_{i j} \qquad ( EQ . 10 )$$

$${\vec{F}}_{c n d , j i} = ( {C}_{H} {s}_{n}^{\frac{1}{4}} {\dot{s}}_{n} ) {\vec{n}}_{i j} = ( 2 {η}_{H} \sqrt{{m}^{⋆} {K}_{n}} {s}_{n}^{\frac{1}{4}} {\dot{s}}_{n} ) {\vec{n}}_{i j} \qquad ( EQ . 11 )$$

$${\vec{F}}_{c n a d h , j i} = \sqrt{8 π Γ {E}^{⋆} {a}^{3}} {\vec{n}}_{i j} \qquad ( EQ . 12 )$$

wherein Kₙ is the normal contact stiffness, sₙ is the normal contact overlap (shown in FIG. 3A), \dot{s}ₙ is the time derivative of sₙ, [symbol] ⱼᵢ is the unit normal vector, a is the radius of contact between the particles 78 or between a particular particle 78 and a boundary 82 (shown in FIG. 3A), E* is the effective Young's Modulus, R* is the effective radius, C_{H} is the normal damping coefficient, m* is the effective mass, and η_{H} is the normal damping ratio for the Hertzian model, which can be defined by:

$$\frac{1}{{E}^{*}} = \frac{1 - {σ}_{1}^{2}}{{E}_{1}} + \frac{1 - {σ}_{2}^{2}}{{E}_{2}} \qquad ( EQ . 13 )$$

$$\frac{1}{{R}^{*}} *= \{ \begin{matrix}\frac{2}{{d}_{p , i}} + \frac{2}{{d}_{p , j}} , & particle - particle contact \\ \frac{2}{{d}_{p}} , & particle - boundary contact\end{matrix} \qquad ( EQ . 14 )$$

$$\frac{1}{{m}^{*}} = \{ \begin{matrix}\frac{1}{{m}_{p 1}} + \frac{1}{{d}_{p 2}} , & particle - particle contact \\ \frac{1}{{m}_{p}} , & particle - boundary contact\end{matrix} \qquad ( EQ . 15 )$$

$${η}_{H} = \frac{\sqrt{5}}{2} η \qquad ( EQ . 16 )$$

In EQ. 13, E₁ and E₂ are Young's moduli of the particles 78 or the particular particle 78 and the boundary 82. In EQ. 14, d_{p,i} and d_{p,j} are the sizes of the particles 78, and dₚ is the size of the particular particle 78 in contact with the boundary 82. In EQ. 15, mₚ₁ and mₚ₂ are the mass of the first particle 78a and the second particle 78b, respectively, and mₚ is the mass of the particular particle 78 in contact with the boundary 82. In EQ. 16, η is the damping ratio, a dimensionless parameter whose value is related to the restitution coefficient ε, which can be given by:

$$ε = \{ \begin{matrix}exp [ − \frac{η}{\sqrt{1 − {η}^{2}}} ( π − {tan}^{− 1} \frac{2 η \sqrt{1 − {η}^{2}}}{1 − 2 {η}^{2}} ) ] , & 0 \leq η < \frac{\sqrt{2}}{2} \\ exp ( − \frac{η}{\sqrt{1 − {η}^{2}}} {tan}^{− 1} \frac{2 η \sqrt{1 − {η}^{2}}}{2 {η}^{2} − 1} ) , & \frac{\sqrt{2}}{2} \leq η < 1 \\ exp ( − \frac{η}{\sqrt{{η}^{2} − 1}} ln \frac{η + \sqrt{{η}^{2} − 1}}{η − \sqrt{{η}^{2} − 1}} ) , & 1 < η\end{matrix} \qquad ( EQ . 17 )$$

wherein the restitution coefficient ε is the user input that represents the particle-particle or particle-boundary interactions. Additionally, effect radius (R*) can be calculated from the normal contact overlap sₙ by:

$${s}_{n} = \frac{{a}^{2}}{{R}^{*}} - {( \frac{2 πΓ {a}^{2}}{{E}^{*}} )}^{\frac{1}{2}} \qquad ( EQ . 18 )$$

wherein Γ is the surface energy.

Additionally, the tangential elastic force [symbol] _{ct,ji} (EQ. 7) consists of the tangential spring force [symbol] _{ct,ji}, the tangential viscous damping force [symbol] _{ctd,ji}, and the frictional force [symbol] _{cf,ji}. The tangential elastic force [symbol] _{ct,ji} can be calculated using the Mindlin-Deresiewicz model, for example:

$${\vec{F}}_{t , ji} = - {μ}_{p} ‖ {\vec{F}}_{c n , j i} ‖ ( 1 - {λ}^{\frac{3}{2}} ) \frac{{\vec{s}}_{τ}}{‖ {\vec{s}}_{τ} ‖} + {{η}_{τ} ( \frac{6 {μ}_{p} {m}^{*} ‖ {\vec{F}}_{cn , ji} ‖}{{s}_{τ , max}} )}^{\frac{1}{2}} {λ}^{\frac{1}{4}} {\dot{\vec{s}}}_{τ} ) \qquad ( EQ . 19 )$$

$$λ = \{ \begin{matrix}1 − \frac{min ( ‖ {\vec{s}}_{τ} ‖ , {s}_{τ , max} )}{{s}_{τ , max}} , & \vert {\vec{s}}_{τ} \vert \leq {s}_{τ , max} \\ 0 , & \vert {\vec{s}}_{τ} \vert > {s}_{τ , max}\end{matrix} \qquad ( EQ . 20 )$$

wherein μₚ is the friction coefficient, η_{τ} is the tangential damping ratio estimated, [symbol] _{τ} is the tangential relative displacement at the contact, [symbol] _{τ} is the tangential component of the relative velocity at the contact, and s_{τ,max} is the maximum relative tangential displacement at which the particles 78 begin to slide. Specifically, μₚ can be given as:

$${μ}_{p} = f ( x ) = \{ \begin{matrix}{μ}_{s} , no sliding at the contact \\ {μ}_{d} , no sliding at the contact\end{matrix} \qquad ( EQ . 21 )$$

wherein μₛ and μ_{d} are the static and dynamic friction coefficients, respectively. The tangential damping ratio may be given as:

$${η}_{τ} = \frac{ln ε}{\sqrt{{ln}^{2} ε + {π}^{2}}} \qquad ( EQ . 22 )$$

The value of the maximum relative tangential displacement s_{τ,max} may be determined by:

$${s}_{τ , max} = {{μ}_{p} ( \frac{1 - {σ}_{1}}{2 - {σ}_{1}} + \frac{1 - {σ}_{2}}{2 - {σ}_{2}} )}^{- 1} {s}_{n} \qquad ( EQ . 23 )$$

wherein σ₁ and σ₂ are the Poisson's ratios of the particles 78 or the particular particle 78 and the boundary 82.

In addition, the eddy lifetime model may be employed to account for particle interaction with turbulence eddies and the local turbulence fluctuation velocity components. In some embodiments, the particles 78 may be tracked using the Lagrange method by solving for individual trajectories using the validated CFPD method. Additionally, in some embodiments, the particles 78 that have escaped from G13 outlets may be considered deposited and/or absorbed in the G13-to-alveoli region. In some embodiments, particle deposition in the patient respiratory system 70 may be quantified using DFs, defined as the mass of the particles 78 deposited in a specific lung region divided by the total mass of the particles 78 entering the mouth.

The in situ model may be further validated. Validation may aid in optimizing simulation of particle trajectories and/or airflow patterns in patient respiratory systems 70 (shown in FIG. 3A). In some embodiments, the in situ model may be validated via matching in vitro particle DFs in the oral/nasal cavities and/or TB tree.

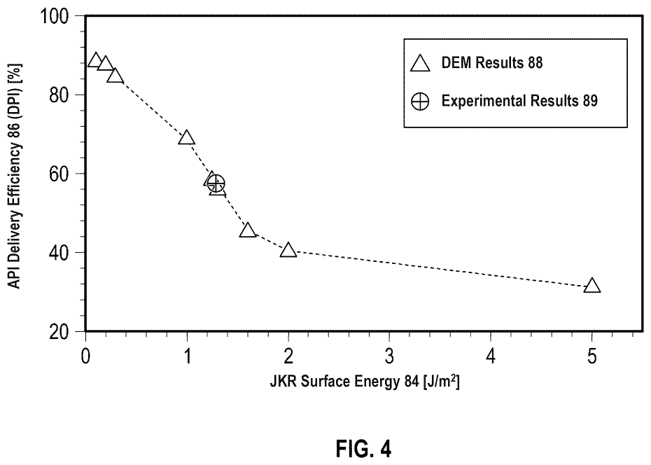

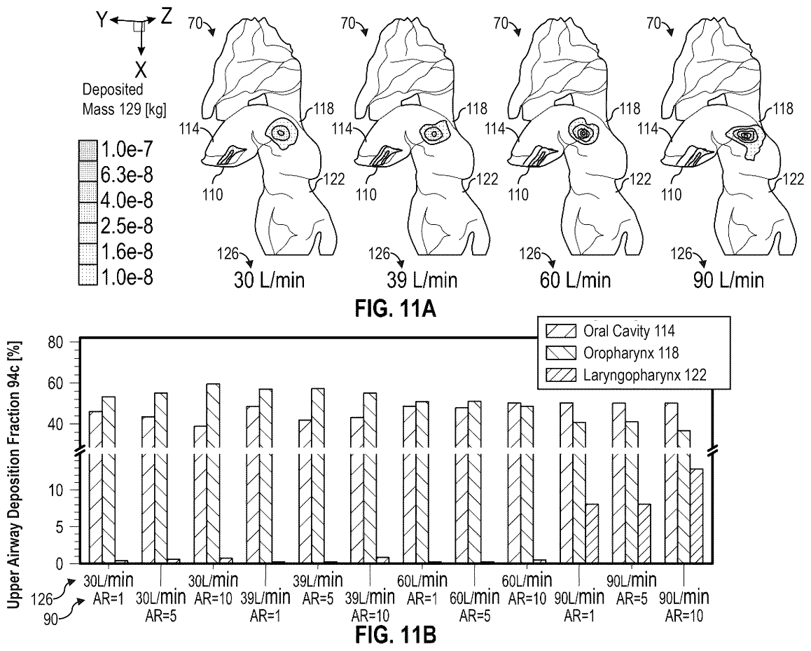

The in situ model may be further calibrated. Calibration may account for surface energy between the particles 78 (e.g., the API particles 78c and the lactose particles 78d) and the wall 20, static friction coefficient, dynamic friction coefficient, predictions of the particle-particle interactions and emitted APSDs, and/or the like. In some embodiments, experimental measurements of the parameters described herein may be obtained or calibrated. In some embodiments, calibrations of friction coefficients and surface energy between the particles 78 and the wall 20 may be performed using numerical simulations. For example, in some embodiments, a range of surface energy values (e.g., from 0.01 to 10 J/m²) may be used in CFD-DEM simulations to match the delivery efficiency of the inhaler 14 (i.e., fractions of drugs emitted from the mouthpiece 34) measured in vitro. As shown in Table 2, employing Qᵢₙ 126 (shown in FIG. 11B)=39 L/min as a representative setup, CFD-DEM simulations were performed with different surface energies between the particles 78 and the wall 20, the friction coefficient between the particles 78, and friction coefficient between the particles 78 and the wall 20 (see Table 2 for the simulation results with different parameter values). The API delivery efficiency 86 of the first inhaler 14a was compared with experimental data documented by the FDA for parameter value calibrations. Determined by best agreements on the API delivery efficiency 86 between DEM results 88 and experimental results 89 (shown in FIG. 4), calibrated parameter values are listed in Table 1 for this example.

TABLE 1
Calibrated DEM properties for API particles 78c and lactose particles 78d.

| | API 78c - | API 78c - | Lactose 78d - | API 78c - | Lactose 78d - |
|---|---|---|---|---|---|
| | API 78c | Lactose 78d | Lactose 78d | Wall 20 | Wall 20 |
| Surface Energy Γ | 43.4e−3 | 47.5e−3 | 13.4e−3 | 1.29 | 1.29 |
| [J/m²] | | | | | |
| Static Friction | 0.7 | 0.7 | 0.7 | 0.5 | 0.5 |
| Coefficient | | | | | |
| Dynamic Friction | 0.7 | 0.7 | 0.7 | 0.5 | 0.5 |
| Coefficient | | | | | |

TABLE 2
Particle (i.e., API particle 78c) delivery efficiency of the first inhaler
14a by CFD-DEM simulations with different parameter values for calibration.

| | | Friction Factor | Friction Factor | | |
|---|---|---|---|---|---|
| | JKR Surface | (Particle 78 - | (Particle 78 - | Rolling | API Delivery |
| | Energy Γ | Particle 78) | Boundary 82) | Resistance | Efficiency 86 |
| ID | [J/m²] | [—] | [—] | [—] | [%] |
| 1 | 0.25 | 0.7 | 0.3 | non-rolling | 95.091 |
| 2 | 0.4 | 0.7 | 0.5 | non-rolling | 94.221 |
| 3 | 0.5 | 0.7 | 0.5 | non-rolling | 88.909 |
| 4 | 1 | 0.7 | 0.5 | non-rolling | 69.091 |
| 5 | 1.25 | 0.7 | 0.5 | non-rolling | 58.971 |
| 6 | 1.3 | 0.7 | 0.5 | non-rolling | 56.793 |
| 7 | 1.6 | 0.7 | 0.5 | non-rolling | 46.169 |
| 8 | 2 | 0.7 | 0.5 | non-rolling | 40.727 |
| 9 | 5 | 0.7 | 0.5 | non-rolling | 31.455 |

In some embodiments, to further determine the JKR surface energy between the particles 78 and the wall 20 (i.e., the JKR particle-wall surface energy 84 (hereinafter the “JKR surface energy 84”)), regressions may be perfumed to correlate the relationship between the API delivery efficiency 86 and the JKR surface energy 84 (see FIG. 4). As shown in FIG. 4, the API delivery efficiency 86 is a linear function of the JKR surface energy 84 when the JKR surface energy 84 is less than 2 J/m². The correlation can be given as:

$$D {E}_{Lactose + API} = - 43.56 {Γ}_{particle - device} + 1 13.4 {Γ}_{particle} ∈ [ 0.4 , 2 ] J / {m}^{2} ) \qquad ( EQ . 24 )$$

As such, Γ_{particle-device}=1.29 J/m². In addition, it can be further concluded that if the JKR surface energy 84 property between the particles 78 and the wall 20 is reduced, the API delivery efficiency 86 is enhanced accordingly.

In some embodiments, CFD simulations of the airflow field in the flow channel 18 and CFPD simulations of pulmonary air-particle flow dynamics may be determined using Ansys Fluent 2020 R2 (Ansys Inc., Canonsburg, PA), or similar. A semi-implicit method for pressure-linked equations (SIMPLE) algorithm may be employed for the pressure-velocity coupling, and a least-squares cell-based scheme may be applied to calculate the cell gradients. A second-order scheme may be employed for pressure discretization. In addition, a second-order upwind scheme may be applied for the discretization of momentum and turbulent kinetic energy. Convergence is defined for continuity, momentum, and supplementary equations when residuals are less than 1.0e-5.

Coupled with CFD simulations of the airflow field in the flow channel 18, DEM simulations may be performed using Ansys Rocky 4.4.3 (Ansys Inc., Canonsburg, PA), or similar. The number of lactose particles 78d may be 7,166, for example. The number of the particles 78 released in the capsule chamber 26 may 1,713,008, for example. In some embodiments, the simulated number of the particles 78 may be one-tenth of the real number of the particles 78 in the capsule 36 to reduce 86% of the computational time and provide similar API delivery efficiency 86 predictions (i.e., less than 5% difference) compared with simulations using the real number of the particles 78 in the capsule 36.

In some embodiments, one or more user-defined functions (UDFs) may be used. The UDFs may include, but are not limited to, measuring emitted APSDs from the orifices of the inhaler 14 (i.e., the inlet 22 and/or the mouthpiece 34) and conversion into particle release maps as the inlet conditions for lung aerosol dynamics simulations; specifying the transient inhalation profile at the mouth; recovering the anisotropic corrections on turbulence fluctuation velocities; modeling the Brownian motion-induced force; storing particle deposition data; and/or the like.

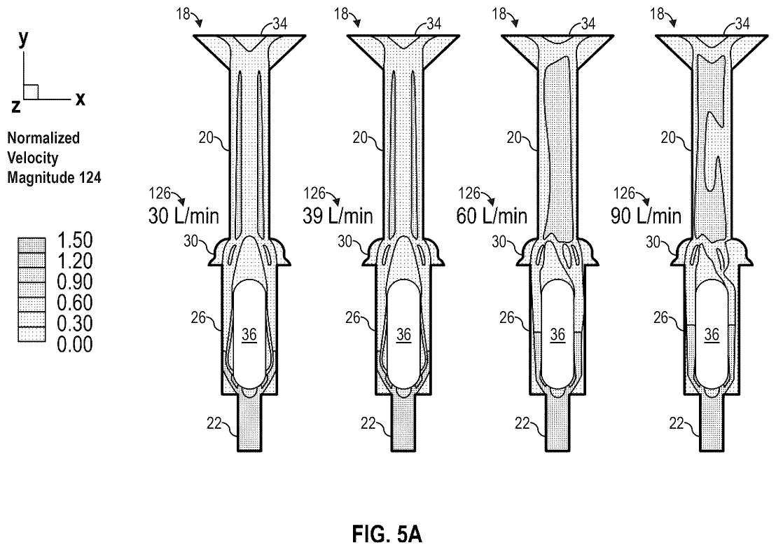

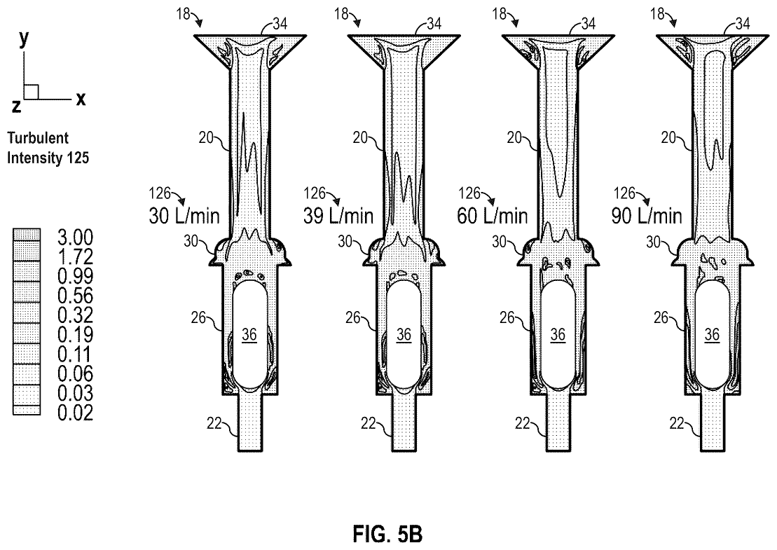

FIGS. 5A and 5B illustrate airflow structure within the flow channel 18 using the in situ model. Distributions of normalized velocity magnitude 124 (∥\vec{V}∥/∥\vec{Vᵢₙ}∥) and turbulence intensity 125 (TI) at four different Qsᵢₙ 126 (i.e., 30, 39, 60, and 90 L/min) are shown in FIGS. 5A and 5B. Specifically, the normalized velocity magnitude 124 contours at plane z=0 are shown in FIG. 5A. It can be observed that the maximum velocity is located adjacent to the bottom region of the capsule 36, due to the narrowed airflow passage with the presence of the capsule 36 and the skewed velocity profiles near a surface of the capsule 36 created by impingement of airflow in the flow channel 18. The velocity contours with Qᵢₙ 126=30 and 39 L/min share similar patterns in the computational domain near the capsule 36. Flow detachments can be found downstream the locations where the airflow impacts the capsule 36. At higher flow rates (Qᵢₙ 126=60 and 90 L/min), flow separations did not occur adjacent to a bottom region of the capsule 36. Instead, separation locations shift further downstream, compared with cases with Qᵢₙ 126=30 and 39 L/min. Indeed, with higher Qᵢₙ 126, the flow momentum after the impaction of the capsule 36 remains higher. Therefore, the flow with higher Qᵢₙ 126 (i.e., 60 and 90 L/min) is able to conquer the viscous dissipation effect, and generate no flow separation near a wall of the capsule 36, compared with the flow with lower Qᵢₙ 126 (i.e., 30 and 39 L/min). Based on the TI 125 comparisons shown in FIG. 5B, higher TI 125 (i.e., TI 125>3) can be observed near a wall of the capsule chamber 26 in cases with higher Qᵢₙ 126 (i.e., 60 and 90 L/min). In contrast, for cases with Qᵢₙ 126=30 and 39 L/min, high TI 125 (TI 125≥300%) can be found only at the lower middle region near the wall of the capsule 36 and the bottom region of the capsule chamber 26. TI 125 is approximately 30% from the top of the capsule chamber 26 to the mouthpiece 34. It can also be observed from FIG. 5B that increasing Qᵢₙ 126 can elongate the high-TI cores as an indicator of stronger turbulence fluctuations.

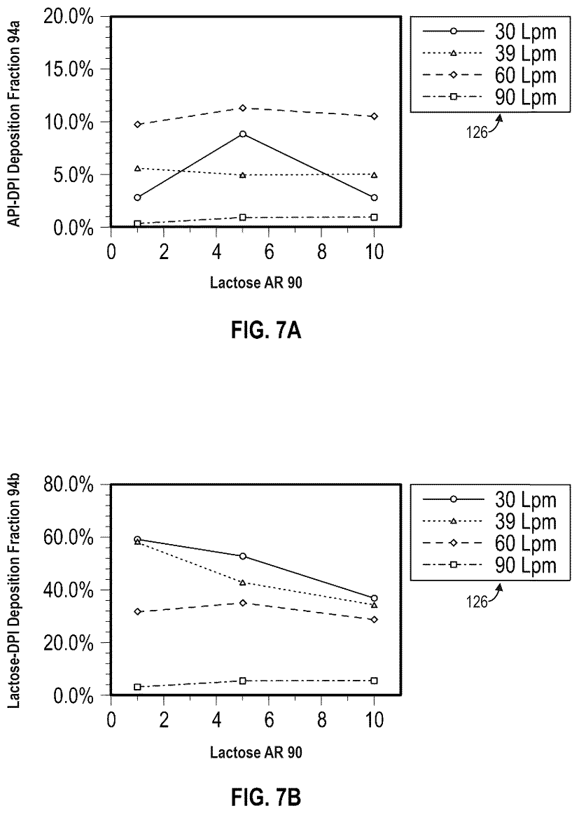

FIGS. 6, 7A, and 7B illustrate deposition of the particles 78 in the flow channel 18 and API delivery efficiency 86 of the first inhaler 14a. For example, localized particle delivery deposition patterns in the flow channel 18 with different Qᵢₙ 126 and AR 90 (hereinafter the “lactose AR 90”) (shown in FIG. 7A) of lactose particles 78d are shown in FIG. 6. Here, lactose AR 90 is used to represent the aspect ratio of lactose particles 78d only (i.e., quasi-spherical API particles 78c). At Qᵢₙ 126=30 and 39 L/min, the “hot spots” of depositions of lactose particles 78d are the surface of the capsule 36 and the wall of the capsule chamber 26 near the bottom opening of the capsule chamber 26. Another concentrated deposition site for lactose particles 78d is the grid 30, especially for spherical lactose particles 78d (lactose AR 90=1). At Qᵢₙ 126=60 or 90 L/min, the number of deposited lactose particles 78d in the first inhaler 14a is less than that in cases with Qᵢₙ 126=30 and 39 L/min. This is due to the more substantial resuspension effect induced by more intense aerodynamic forces (e.g., the drag force) acting on the deposited particles 78 generated by higher airflow velocities. As a result, more deposited lactose particles 78d and API particles 78c may be resuspended and transported along with the airflow downstream and exit the mouthpiece 34. It can also be observed that the shape of lactose particles 78d may have a noticeable influence on lactose delivery deposition patterns in the first inhaler 14a. Specifically, at Qᵢₙ 126=30 and 39 L/min, the deposited lactose particles 78d in the capsule chamber 26 decreases with the increasing lactose AR 90 (also see FIG. 7B for the total DF_{lactose-DPI} 94b). Such an observation indicates that lactose particles 78d that are more elongated can deliver more of the particles 78 into the mouth than the lactose particles 78d with more isotropic shapes and the same particle volume. At Qᵢₙ 126=60 L/min, with an increase in lactose AR 90, fewer lactose particles 78d are trapped in the capsule chamber 26 but are deposited more downstream in the extending tube. Therefore, relatively elongated lactose particles 78d can transport further downstream compared with more isotropic-shape particles with the same volume. This may be due (1) the elongated particles 78 are able to follow the airflow streams better than spherical particles with the same volume; and/or (2) with the same particle volume, deposited elongated particles 78 may be easier to resuspend than particles 78 in more isotropic shapes. Compared with cases at Qᵢₙ 126=60 L/min, similar particle delivery deposition patterns can be observed in the cases with Qᵢₙ 126=90 L/min. However, at this flow rate, most of the lactose particles 78d were emitted with the strongest convection effect generated by the highest Qᵢₙ 126, making the impact of lactose AR 90 on particle delivery deposition patterns not evident for Qᵢₙ 126=90 L/min. Thus, in using the in situ model, FIG. 6 indicates that with the same particle volume, lactose particles 78d that are more elongated can be better at evading collision with the wall 20 and more accessible to be resuspended by the airflow after deposition, which leads to less deposition in the flow channel 18 than expected from particles 78 with more isotropic shapes.

For API particles 78c, FIG. 6 shows that most API particles 78c are deposited in the capsule chamber 26, capsule surface, and the cap wall above the grid 30 for cases with Qᵢₙ 126=30 and 39 L/min. At Qᵢₙ 126=60 L/min, the number of API particles 78c deposited on the cap wall and surface of the capsule 36 is reduced compared with 30 and 39 L/min cases, while more API particles 78c are deposited at the bottom of the capsule chamber 26. At Qᵢₙ 126=90 L/min, most API particles 78c were emitted through the mouthpiece 34 as there are few particles trapped either inside the capsule chamber 26 of the cap wall.

DFs 94, which may include DFs of API particles 78c (i.e., DF_{API-DPI} 94a) and lactose particles 78d (i.e., DF_{lactose-DPI} 94b), in the flow channel 18 are presented in FIGS. 7A and 7B with multiple Qᵢₙ 126 and lactose AR 90. It can be observed from FIG. 7A that the in situ model may determine that the influence of lactose AR 90 is not significant on DF_{API-DPI} 94a for cases with Qᵢₙ 126=60 L/min and 90 L/min since the turbulence dispersion effect is relatively more dominant. However, at Qᵢₙ 126=30 L/min, API deposition in the inhaler 14 reaches the maximum (i.e., DF_{API-DPI} 94a equal to 8.8%, with lactose AR 90 equal to 5). This is possibly due to the combined effect of the variations in the easiness of deposition and resuspension with the lactose AR 90 changes.

The impact of Qᵢₙ 126 on DF_{API-DPI} 94a is also shown in FIG. 7A, without a unified trend. Specifically, when lactose AR 90=1 or 10, the increase in Qᵢₙ 126 from 30 L/min to 60 L/min leads to the increase of DF_{API-DPI} 94a. With the further increase in Qᵢₙ 126 from 60 L/min to 90 L/min, DF_{API-DPI} 94a decreases. Such non-monotonic trends are possibly due to the following mechanisms. Specifically, at lower Qᵢₙ 126, even though the convection effect is weaker than the high flow rate condition, the turbulent dispersion effect is also weaker (see FIGS. 5A and 5B). As a result, fewer API particles 78c may be deposited in the capsule chamber 26 compared with 39 and 60 L/min cases. At high Qᵢₙ 126 (e.g., 60 L/min), the TI 125 in the capsule chamber 26 can reach as high as 300%, which leads to a high DF_{API-DPI} 94a in the bottom region of the capsule chamber 26 (see FIG. 6 for the 60 L/min cases). Meanwhile, the deposited API particles 78c in that region may not be sufficiently resuspended by the aerodynamic forces, as the convection effect in the capsule chamber 26 at 60 L/min is not strong enough. As Qᵢₙ 126 increases to 90 L/min, the convection effect becomes more dominant and sufficiently strong to overcome the adhesion between the API particles 78c and the wall 20. Therefore, API particles 78c can resuspend more and be carried by the airflow to the mouthpiece 34, which results in the decrease in DF_{API-DPI} 94a at Qᵢₙ 126=90 L/min compared with Qᵢₙ 126=60 L/min.

Referring to FIG. 7B, the in situ model illustrated lactose DFs in the inhaler 14 (i.e., DF_{lactose-DPI} 94b) may be influenced by both Qᵢₙ 126 and lactose AR 90. DF_{lactose-DPI} 94b decreases significantly from 59% to approximately 6.0% as Qᵢₙ 126 increases from 30 to 90 L/min with spherical lactose particles 78d (i.e., lactose AR 90=1). Such trends imply that the turbulence has a weaker effect on the DF_{lactose-DPI} 94b than DF_{API-DPI} 94a, since lactose particles 78d are much larger than API particles 78c. Although the same trend between DF_{lactose-DPI} 94b and Qᵢₙ 126 is observed for elongated lactose particles 78d (i.e., lactose AR 90=10) with the same particle volume, DF_{lactose-DPI} 94b only decreases by 8.6% as the flow rate increases from 30 to 60 L/min. The decrease in DF_{lactose-DPI} 94b is less significant for the most elongated lactose particles 78d (i.e., lactose AR 90=10) is because that the flow exerts a smaller drag force on the elongated particles than the spherical particles with the same equivalent diameter, which means that the elongated particles may be more likely to be emitted through the mouthpiece 34. Specifically, when transported by the airflow, the major axis of the elongated particles is along the same direction of the airflow direction. Thus, the drag force acting on the elongated particles may be reduced compared with spherical particles.

For the effect of lactose AR 90 on DF_{lactose-DPI} 94b in the inhaler 14, the in situ model in FIG. 7B illustrates that at Qᵢₙ 126=30 and 39 L/min, DF_{lactose-DPI} 94b decreases from approximately 50% to 35%, with the increase in lactose AR 90. The influence of lactose AR 90 on DF 94 is not evident when the flow rate reaches 60 and 90 L/min, as the total DF_{lactose-DPI} 94b fluctuates around 30% (i.e., Qᵢₙ 126=60 L/min) and 4% (i.e., Qᵢₙ 126=90 L/min), respectively. The non-monotonic relationship between DF_{lactose-DPI} 94b and lactose AR 90 can also be due to combined influences from the variations in the easiness of deposition and resuspension with the lactose AR 90 changes.

As illustrated in FIGS. 7A and 7B, particle resuspension, in addition to or in lieu of using the idealized 100% trapped in the wall 20, may enable prediction of the more complex and realistic lactose shape effect on API particle 78c and lactose particle 78d transport and deposition. Overall, a high Qᵢₙ 126 (e.g., 90 L/min) and more elongated lactose particles 78d (i.e., lactose AR 90=10) can potentially reduce the loss of API particles 78c and lactose particles 78d in the inhaler 14, thereby enhancing the API delivery efficiency 86 to the human mouth opening 110 (shown in FIG. 10A).

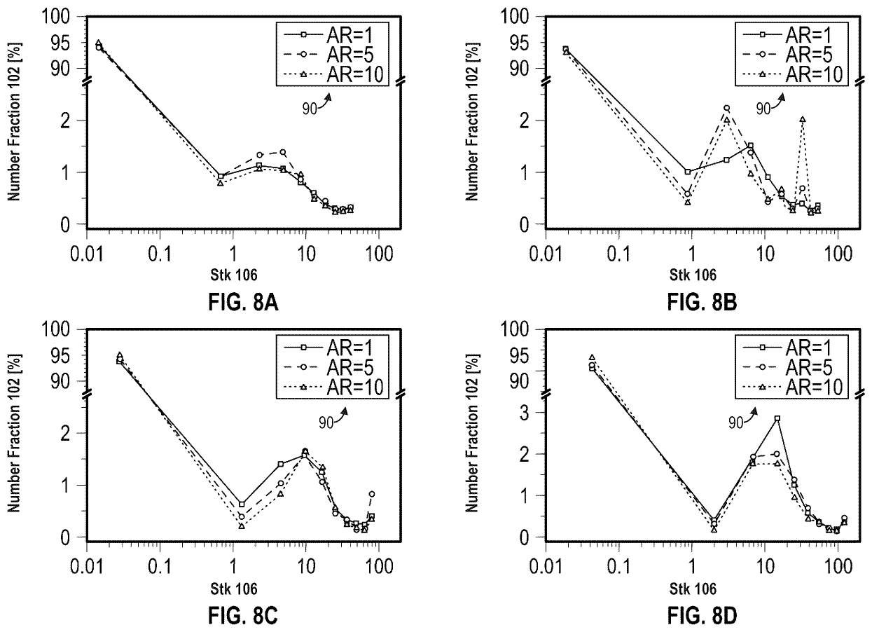

FIGS. 8A-8D illustrate the effects of particle shape and Qᵢₙ 126 on emitted APSDs using the inhaler 14. The number fraction (NF) 102 is defined as the number of the particles 78 within a specific size being divided by the total number of the particles 78 emitted, including both API particles 78c and lactose particles 78d. The Stokes number (Stk) 106 is calculated based on outlet airflow mean velocity of inhaler 14. In general, when Stk 106 is less than 1, the particles 78 can follow the airflow path naturally. At Qᵢₙ 126=30 L/min, similar APSD patterns can be observed for Stk 106 from 7 to 40 (i.e., particles 78 with dₚ from 50 μm to 114 μm) for different lactose AR 90 (shown in FIG. 8A). Moreover, the most elongated lactose particles 78d (i.e., lactose AR 90=10) provided the highest NF 102 (i.e., 95%) for small particles 78 (i.e., dₚ≤4.3 μm), which are mostly API particles 78c. The case with lactose AR 90=10 predicts lower NF 102 for small particles due to the high NF 102 of particles 78 with dₚ=90 μm predicted (shown in FIG. 8B). At Qᵢₙ 126=60 and 90 L/min (shown in FIGS. 8C and 8D), using more elongated lactose particles 78d (i.e., lactose AR 90=10) predicted higher NF 102 for small particles 78 (i.e., dₚ≤4.3 μm) than using lactose particles 78d with less anisotropic shapes (i.e., lactose AR 90=1 and 5).

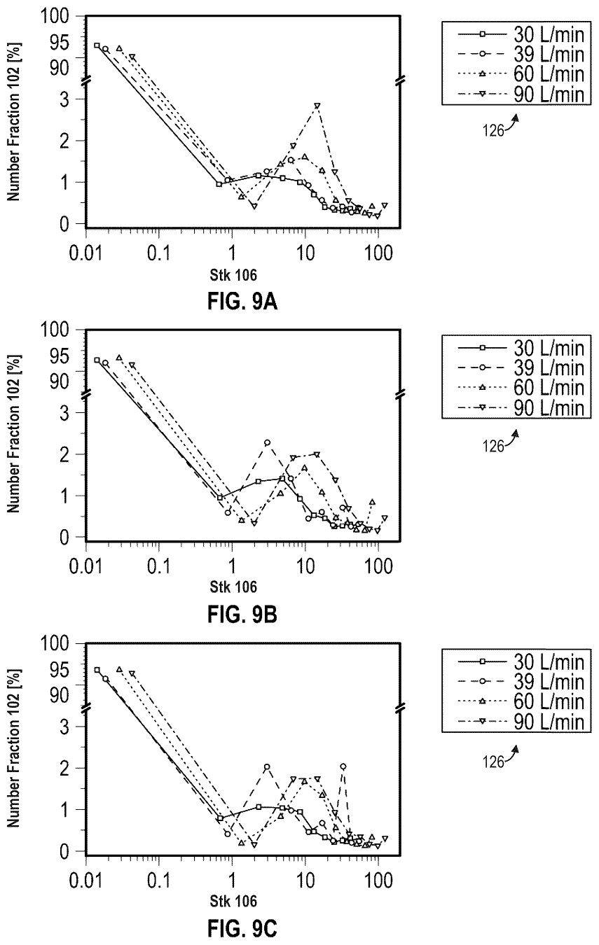

The effect of Qᵢₙ 126 on emitted APSDs is presented in FIGS. 9A-9C. NF_{API} 102 (i.e., dₚ≤4.3 μm) are at a high-level ranging from 92% to 96% for all Qᵢₙ 126 values. Specifically, using spherical lactose particles 78d with lactose AR 90=1 (shown in FIG. 9A), NF_{API} 102 decreases with the decrease in Qₙ, since more lactose particles 78d with large size (i.e., dₚ>30 μm) were emitted at a higher flow rate (shown in FIG. 6). For particles 78 (10 μm<dₚ<60 μm), NF_{lactose} 102 increases with the increase in Qᵢₙ 126, which is consistent with the observations in FIGS. 6, 7A-7B, and 8A-8D. Especially for Qᵢₙ 126=90 L/min, the NF 102 of particles 78 with dₚ=40 μm reaches 2.7%. With elongated lactose particles 78d (i.e., lactose AR 90=5) shown in FIG. 9B, cases with all Qᵢₙ 126 values predict a similar trend of APSDs as the cases using spherical lactose particles 78d (i.e., lactose AR 90=1) (shown in FIG. 9A). Specifically, Qᵢₙ 126=90 L/min case predicts the lowest NF_{API} 102 (i.e., 93.1%) for all Qᵢₙ 126 values. For particles 78 with dₚ>20 μm, high Qᵢₙ 126 cases (i.e., 60 and 90 L/min) generate higher NF_{lactose} 102 than the case with low flow rate (i.e., Qᵢₙ 126=30 L/min). In contrast, when lactose AR 90=10 (shown in FIG. 9C), Qᵢₙ 126=39 L/min leads to the lowest NF_{API} 102 (dₚ≤4.3 μm) compared with Qᵢₙ 126=30, 60 and 90 L/min cases. For all four Qᵢₙ 126 setups, NF_{lactose} 102 (dₚ>20 μm), high Qᵢₙ 126 cases (i.e., 39, 60, and 90 L/min) tend to generate higher NF_{lactose} 102 than the case with low flow rate (i.e., Qᵢₙ 126=30 L/min).

The inspiratory airflow structures at the sagittal plane y=0 for the patient respiratory system 70 (shown in FIG. 3A) are shown in FIGS. 10A and 10B. It should be noted that the human mouth opening 110 has the same elliptic shape as the mouthpiece 34 of the inhaler 14. The highest flow velocity 127 occurs at the human mouth opening 110 due to the narrowed human mouth opening 110 as shown in FIG. 10A. The turbulent kinetic energy (TKE) 128 visualized in FIG. 10B also demonstrates an increasing turbulence fluctuation in the oral cavity 114 and oropharynx 118 with the increase in Qᵢₙ 126.

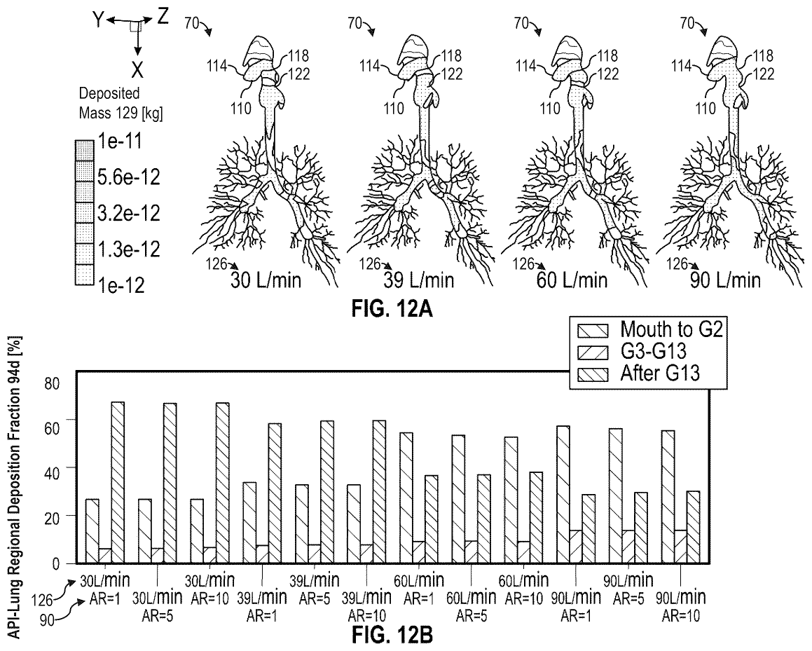

FIGS. 11A and 11B illustrate lactose delivery deposition patterns (shown by deposited mass 129) and DFs_{upper airway} 94c in an upper portion (i.e., an upper airway) of the patient respiratory system 70 at different Qsᵢₙ 126 using the inhaler 14. To investigate how Qᵢₙ 126 and lactose AR 90 can influence lung depositions of lactose particles 78d and API particles 78c, localized delivery deposition patterns of lactose particles 78d (lactose AR 90=1) and its RDFs 94 in the airway model are provided at different Qsᵢₙ 126 (i.e., 30, 39, 60, and 90 L/min) with lactose AR 90=1, 5, and 10. FIGS. 12A and 12B illustrate lung deposition patterns of API particles 78c (i.e., drug delivery deposition patterns) and RDF_{API-lung}94d with different Qsᵢₙ 126 and lactose ARs 90, respectively. The emitted APSDs from the inhaler 14 with specific Qᵢₙ 126 and lactose AR 90 (shown in FIGS. 8A-8D and 9A-9C) were applied as the mouth inlet conditions for the particle tracking in the patient respiratory systems 70.

Based on the lung deposition data predicted using the in situ model, all the lactose particles 78d are trapped in the oral cavity 114, oropharynx 118, and laryngopharynx 122, despite Qᵢₙ 126 and lactose AR 90 variations. The lactose particles 78d deposited on the tongue (i.e., in the oral cavity 114) are mainly due to the inertial impaction of the mouth jets shown in FIG. 10A and particle gravitational sedimentation, which are the two dominant deposition mechanisms for lactose particles 78d with dₚ>50 μm. Other deposition locations for lactose particles 78d are at the posterior of the oropharynx 118 and laryngopharynx 122. This is due to the impaction of the mouth jet after striking the tongue (i.e., the oral cavity 114). For the RDF 94 of lactose particles 78d (RDF_{lactose-lung} 94), several observations can be made based on the results shown in FIG. 11B (i.e., (1) at Qᵢₙ 126=30 and 39 L/min, the DF_{lactose-oral cavity} 94 decreases with the increase in lactose AR 90, while at Qᵢₙ 126=60 and 90 L/min, lactose AR 90 has negligible influence on DF_{lactose-oral cavity} 94; (2) at low Qᵢₙ 126=30 L/min, the DF_{lactose-oropharynx} 94 increases with the increase in lactose AR 90, while at Qᵢₙ 126=39, 60 and 90 L/min, DF_{lactose-oropharynx} 94 decreases with lactose AR 90; and (3) DF_{lactose-laryngopharynx} 94 increases with the increase in lactose AR 90 for all Qᵢₙ 126). These observations demonstrate that for lactose particles 78d with the same volume, relatively elongated lactose particles 78d (i.e., lactose AR 90=10) can follow the mainstream of the airflow better than spherical lactose particles 78d (i.e., lactose AR 90=1) and deposit more downstream in the upper airway. However, due to the large size (i.e., dₚ>50 μm) of the lactose particles 78d, the lactose particles 78d were not able to reach the trachea and beyond.

For API deposition comparisons in patient respiratory systems 70, FIGS. 12A and 12B illustrate that with the increase in Qᵢₙ 126, more API particles 78c are deposited in the oropharynx 118, glottis 130, trachea, and G1-G13 due to the enhanced inertia impaction effects. For example, with spherical lactose particles 78d (i.e., lactose AR 90=1), when the Qᵢₙ 126 increases from 30 to 90 L/min, the DF 94 of API particles 78c in the upper airway (i.e., from mouth to G2) increases from 26.6% to 57.3% (see FIG. 12B). Moreover, the stronger laryngeal jet effect at 90 L/min also results in the highest DF 94 of API particles 78c in the G0-G1 region (i.e., 8.8%) compared with 4.1% at 30 L/min, 5.0% at 39 L/min, and 6.0% at 60 L/min (see FIG. 12B). A high Qᵢₙ 126 not only leads to high DF 94 of API particles 78c in the upper airway (i.e., from mouth to G2), which may not be optimal in terms of API delivery efficiency 86, but may also reduce the DF 94 of API particles 78c in the lower airway (i.e., after G13) and/or lower the API delivery efficiency 86. For example, with spherical lactose particles 78d (i.e., lactose AR 90=1), the DF_{API} 94 in G13-to-alveoli region decreases by 38.2% (i.e., more than half) when Qᵢₙ 126 increases from 30 to 90 L/min. In terms of the effect of lactose AR 90 on the DF 94 of API particles 78c in the airway, FIG. 12B shows that at the same Qᵢₙ 126, lactose AR 90 has little effect on the API RDFs 94 in all three regions. To quantify the API delivery efficiency 86 to the designated lung sites for deeper-airway COPD and/or asthma treatment, overall DPI-airway API delivery efficiency 86 (ψ) is defined (see Table 3 for the definition of ψ) and calculated. The ψ values with different Qᵢₙ 126 and lactose ARs 90 are listed in Table 3. The result demonstrates that low Qᵢₙ 126 (i.e., 30 L/min) is favored to achieve the higher overall API delivery efficiency 86 using the first inhaler 14a, and Qᵢₙ 126 is the dominant factor on the API RDF 94 after G13 compared with the particle shape of lactose particles 78d.

TABLE 3
The overall DPI-airway API delivery efficiency
86 (ψ)* vs. lactose AR 90 and Qᵢₙ 126.

| | | | | |
|---|---|---|---|---|
| Lactose AR 90 | 30 L/min | 39 L/min | 60 L/min | 90 L/min |

First inhaler 14a

| | | | | |
|---|---|---|---|---|
| 1 | 65.0% | 54.8% | 32.9% | 28.6% |
| 5 | 60.7% | 56.0% | 32.9% | 29.4% |
| 10 | 64.7% | 56.3% | 33.7% | 30.0% |

second inhaler 14b

| | | | | |
|---|---|---|---|---|
| 1 | 59.3% | 55.2% | 34.1% | 28.0% |

$$* ψ = \frac{Deposited API after G 13}{Total amount of API injected info the DPI} \times 100 \% = ( 1 - {DF}_{API - DPI} ) D {F}_{API - after G 13}$$

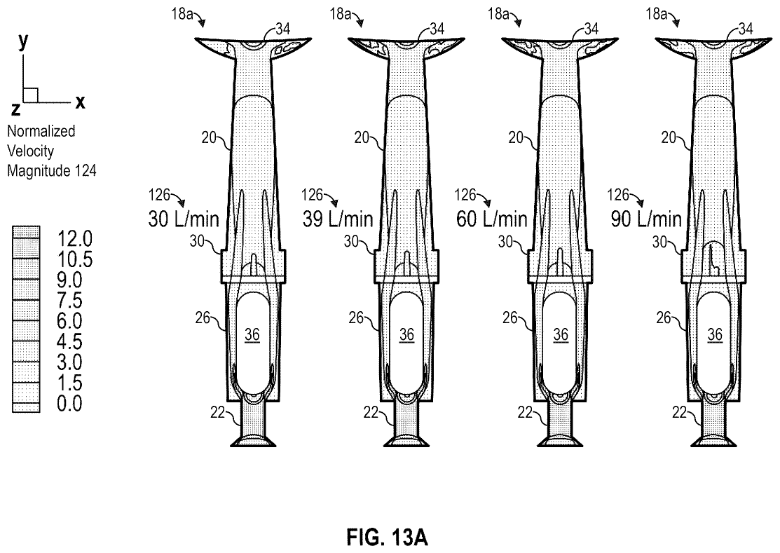

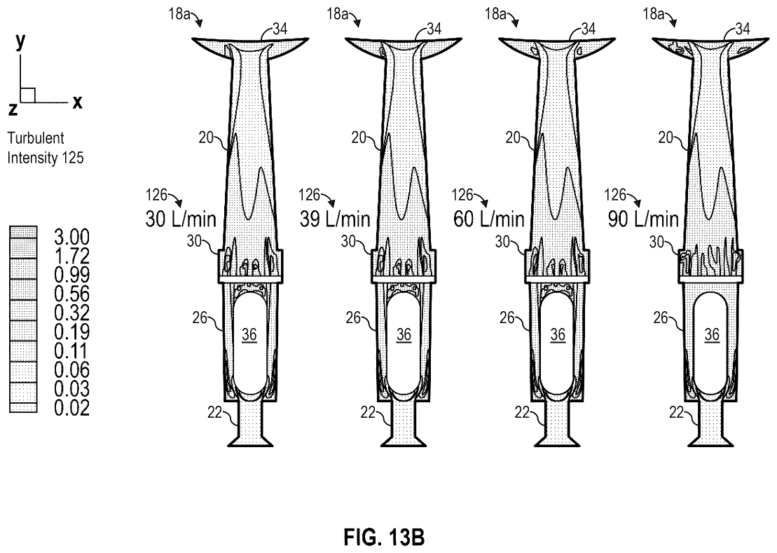

The in situ model may be used to assess the comparability of inhalers (e.g., the inhaler 14 shown in FIGS. 1A and 1B). To evaluate the comparability of airflow fields between inhalers, airflow characteristics may be evaluated. For example, FIGS. 13A and 13B illustrate a prior art flow channel 18a of a prior art inhaler (not shown) with a different Qᵢₙ 126. This is in contrast to the variations of flow separation locations with Qᵢₙ 126 in the flow channel 18 (shown in FIG. 5A) of the present disclosure. The normalized velocity magnitude 124 contours in the prior art flow channel 18a shown in FIG. 13A are similar and less influenced by Qᵢₙ 126. Specifically, no flow separation exists near the bottom of the capsule 36. In addition, the capsule chamber 26 is a straight pipe with a constant diameter for the first inhaler 14a, while the diameter of the capsule chamber 26 of the second inhaler 14b increases gradually in the mainstream direction. Hence, the reverse pressure gradient is less in the capsule chamber 26 than in the first inhaler 14a, which is sufficiently low and avoids the generation of flow separation at all Qᵢₙ 126. As shown in FIG. 13B, the difference in TI distribution is less noticeable among the four cases with different Qᵢₙ 126 in the second inhaler 14b than in the first inhaler 14a (shown in FIG. 5B). Furthermore, it can be observed that the TI 125 near the capsule bottom region increases with the increase in Qᵢₙ 126, indicated by the more extended high-TI cores with the potentially higher turbulence dispersion with the higher Reynolds number. The differences in airflow patterns and geometric designs between the flow channels 18 of inhalers 14 can potentially influence the comparability of particle transport, interaction, and deposition, discussed in the following sections.

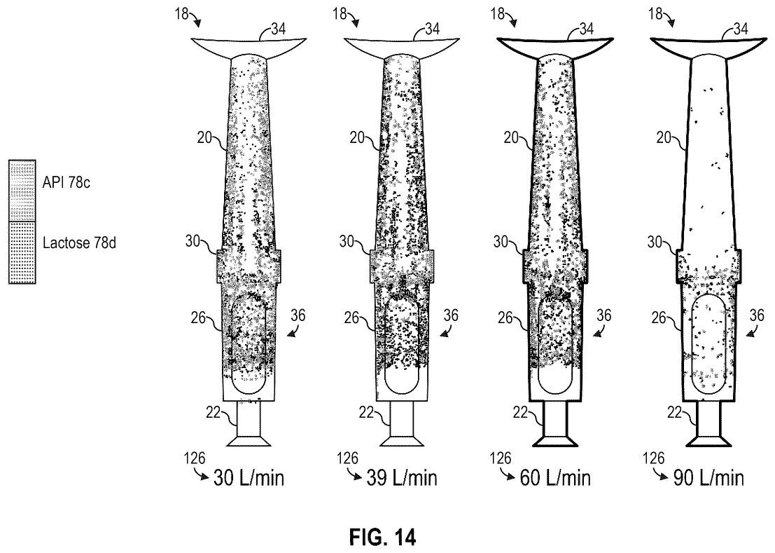

Particle delivery deposition patterns in the second inhaler 14b with different Qᵢₙ 126 and lactose AR 90=1 are shown in FIG. 14. Similar to the deposition patterns in the first inhaler 14a (shown in FIG. 6), when 30 L/min≤Qᵢₙ 126≤60 L/min, both API and lactose depositions scattered in the capsule chamber 26 and the flow channel 18 downstream to the grid 30. When Qᵢₙ 126=90 L/min, the number of particles deposited is significantly reduced, and the majority of particles were emitted from the mouthpiece 34. However, two main differences in the particle delivery deposition patterns can be found between the CFD-DEM results in the inhalers 14 (i.e., (1) for the second inhaler 14b with 30 L/min≤Qᵢₙ 126≤60 L/min, more API particles 78c and lactose particles 78d deposited in the flow channel 18 downstream to the grid 30 than in the first inhaler 14a, since the cone-shape of the flow channel 18 may increase the chance for the particles 78 hitting the wall 20; and (2) compared with the case of the first inhaler 14a with Qᵢₙ 126=60 L/min (shown in FIG. 6), fewer particle depositions are located at the bottom region of the capsule chamber 26 in the second inhaler 14b (shown in FIG. 14). The TI 125 in the bottom region of the capsule chamber 26 of the second inhaler 14b may be lower than that of the first inhaler 14a, hence fewer deposition is induced by the turbulent dispersion.

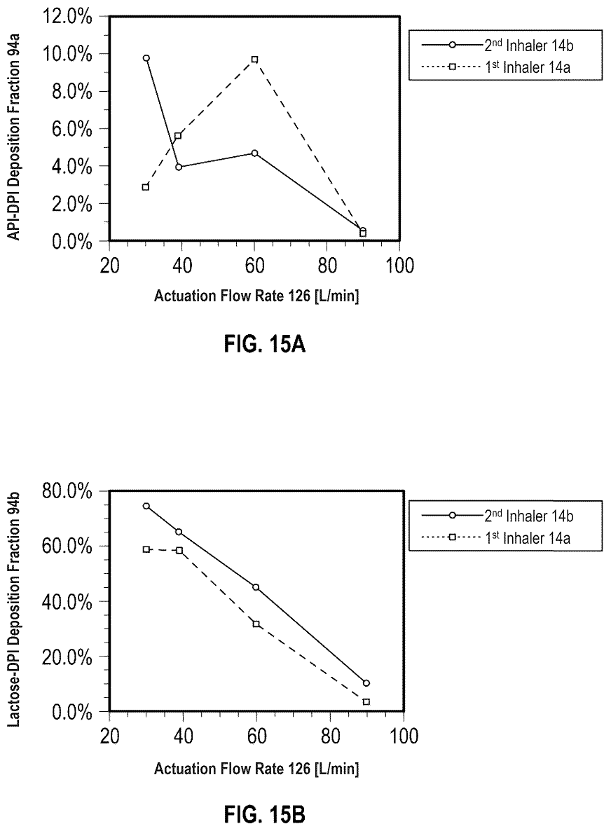

To assess the comparability of the inhalers 14 on API delivery efficiency 86, comparisons of DFs 94 of both API particles 78c and lactose particles 78d in between the flow channels 18 of the inhalers 14 are presented in FIGS. 15A and 15B. FIG. 15A shows that the second inhaler 14b has more API depositions in the device than the first inhaler 14a at Qᵢₙ 126=30 L/min, and it has fewer API depositions in the device than the first inhaler 14a at Qᵢₙ 126=39, 60, and 90 L/min. It indicates that at a relatively higher Qᵢₙ 126, the second inhaler 14b design has a relatively higher API delivery efficiency 86 than the first inhaler 14a. It is worth mentioning that at Qᵢₙ 126=90 L/min, DF_{API} is very close in percentage between the inhalers 14, which indicates that the flow convection effect is strong enough to overcome the JKR surface energy 84 between API particles 78c and the walls 20 of inhalers 14 with different designs. Furthermore, FIG. 15B compares DF_{lactose} 94 in between the inhalers 14 for lactose particles 78d with lactose AR 90=1. It can be observed that both the inhalers 14 show that the DF_{lactose} 94 decreases with Qᵢₙ 126. The first inhaler 14a predicts relatively lower DF_{lactose} 94 than the second inhaler 14b, indicating higher lactose delivery efficiency (not shown). This could be due to the different structural designs of the inhalers 14. Specifically, more lactose particles 78d are deposited on the wall 20 of the flow channel 18 and the grid 30 in the second inhaler 14b than in the first inhaler 14a. Therefore, using the in situ models, determinations may be made that the performance of the second inhaler 14b at Qᵢₙ 126=90 L/min on both API delivery efficiency 86 and lactose delivery efficiency (not shown) are close to the first inhaler 14a. However, at Qᵢₙ 126=30, 39, and 60 L/min, the performances of the inhalers 14 are not very similar.

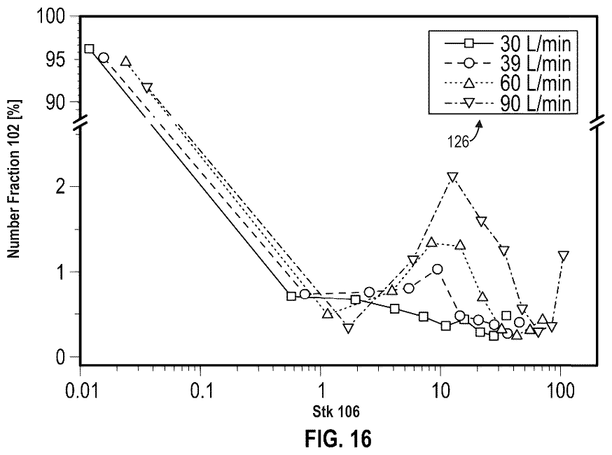

To further evaluate the similarity between the inhalers 14, FIG. 16 illustrates emitted APSDs from the second inhaler 14b with different Qᵢₙ 126. By comparing the APSD predicted by the second inhaler 14b (shown in FIG. 16) and the first inhaler 14a (shown in FIG. 9A) for 30 L/min≤Qᵢₙ 126≤90 L/min, two observations can be made. First, in general, similar APSDs may be generated using both the inhalers 14 for Qᵢₙ 126 ranges from 30 L/min to 90 L/min, which indicates that the second inhaler 14b has a high potential to show comparability. Second, the second inhaler 14b, however, predicts a slightly higher NFs 102 for small particles 78 (i.e., API particles 78c), and lower NFs 102 for large particles 78 (i.e., lactose particles 78d).

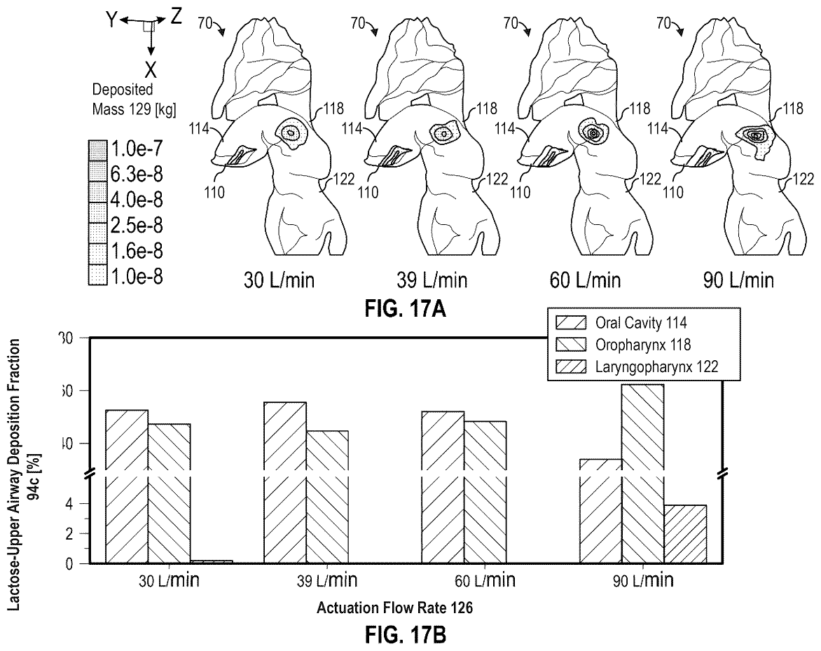

The similarity between the inhalers 14 in airway depositions may be evaluated by comparing the lactose and API (i.e., drug) delivery deposition patterns and RDFs 94 in the patient respiratory system 70. FIG. 17A shows the lactose delivery deposition pattern using the second inhaler 14b. Like the predicted lung deposition data using the first inhaler 14a (shown in FIG. 11A), all the lactose particles 78d were deposited in the upper airway (i.e., the mouth to throat region), due to the dominant inertial impaction and gravitational sedimentation effects for relatively large lactose particles 78d. Using the second inhaler 14b, the deposition in the oral cavity 114 also concentrates on the tongue (i.e., in the oral cavity 114) due to the gravitational sedimentation of large particles 78. The unpreferred deposition on the tongue can be reduced by minimizing the angle between the axial direction of the second inhaler 14b and the centerline of the passage of the oral cavity 114. The rest of the lactose particles 78d carried by the airflow impacted the oropharynx 118 and deposited. As Qᵢₙ 126 increases in the second inhaler 14b, the deposition concentration of lactose particles 78d in the oropharynx 118 also increases due to the more substantial inertial impaction effect, which is similar to the cases using the first inhaler 14a. When comparing the RDFs 94 of lactose particles 78d in the patient respiratory system 70 upon using the first inhaler 14a and the second inhaler 14b (shown in FIG. 11B and FIG. 17B, respectively), lung deposition using the second inhaler 14b has a higher DF_{lactose-oral cavity} 94 than DF_{lactose-oropharynx} 94. In comparison, the resultant depositions of the first inhaler 14a have a lower DF_{lactose-oral cavity} 94 than DF_{lactose-oropharynx} 94 at 30 L/min≤Qᵢₙ 126≤60 L/min. The reason for this difference is the difference in emitted APSD generated by the inhalers 14. Specifically, the second inhaler 14b generates a higher percentage of large lactose particles 78d (i.e., dₚ≥70 μm) than the first inhaler 14a (shown in FIGS. 9A-9C and 16). Hence, when the Qᵢₙ 126 is not sufficiently high to generate a dominant convection effect, the gravitational sedimentation effect will lead to more depositions for the particle distributions with more particles larger than 70 μm. At Qᵢₙ 126=90 L/min, the second inhaler 14b case predicts 16.5% lower in DF_{lactose-oral cavity} 94 and 20.3% higher in DF_{lactose-oropharynx} 94 than the first inhaler 14a case, even though the second inhaler 14b generates 10.2% more large lactose particles 78d (i.e., dₚ≥70 μm) than the first inhaler 14a. This difference could possibly be induced by (1) the dominant convection effect induced higher inertial impaction effect in the oropharynx 118, and (2) the different designs of the mouthpiece 34 between the first inhaler 14a and the second inhaler 14b (shown in FIGS. 1A and 1B, respectively), which leads to different particle injection area at the human mouth opening 110.

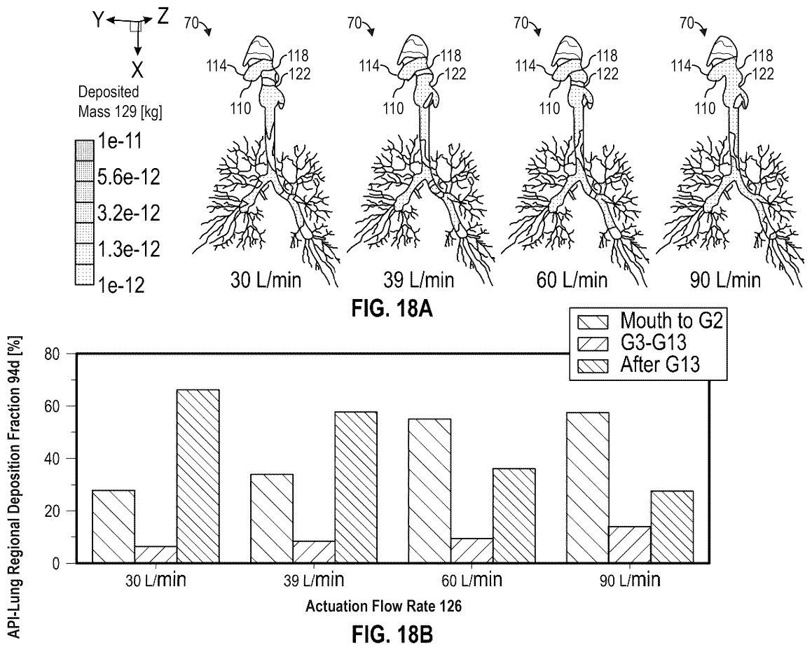

The deposition patterns and RDFs 94 of API particles 78c in the patient respiratory system 70 using the second inhaler 14b are shown in FIGS. 18A and 18B and comparable to the API deposition of the first inhaler 14a shown in FIGS. 12A and 12B. The API lung deposition predicted the second inhaler 14b agrees with the results predicted in the first inhaler 14a cases well (shown in FIGS. 12A and 12B with lactose AR 90=1). The differences in regional lung DF_{API} 94 for all three airway regions between the inhalers 14 are within 2.0% at 30 L/min≤Qᵢₙ 126≤90 L/min. To examine the overall device-airway API delivery efficiency 86, ψ is also calculated for the second inhaler 14b and listed in Table 3. The ψ comparisons between the inhalers 14 using spherical lactose particles 78d (lactose AR 90=1) demonstrate that ψ generated from the second inhaler 14b has a good agreement with the first inhaler 14a at Qsᵢₙ 126 from 30 to 90 L/min. Specifically, at 39 L/min≤Qᵢₙ 126≤90 L/min, the difference in ψ between the inhalers 14 is less than 1.5%. Only at the low Qᵢₙ 126 (i.e., 30 L/min), there is a slightly higher difference in ψ between two cases (5.7%) due to the relatively lower API delivery efficiency 86 of the second inhaler 14b at Qᵢₙ 126=90 L/min. Therefore, using the in situ models, the determination may be made that the second inhaler 14b has a satisfactory agreement with the first inhaler 14a in terms of the general DPI-airway API delivery efficiency 86.

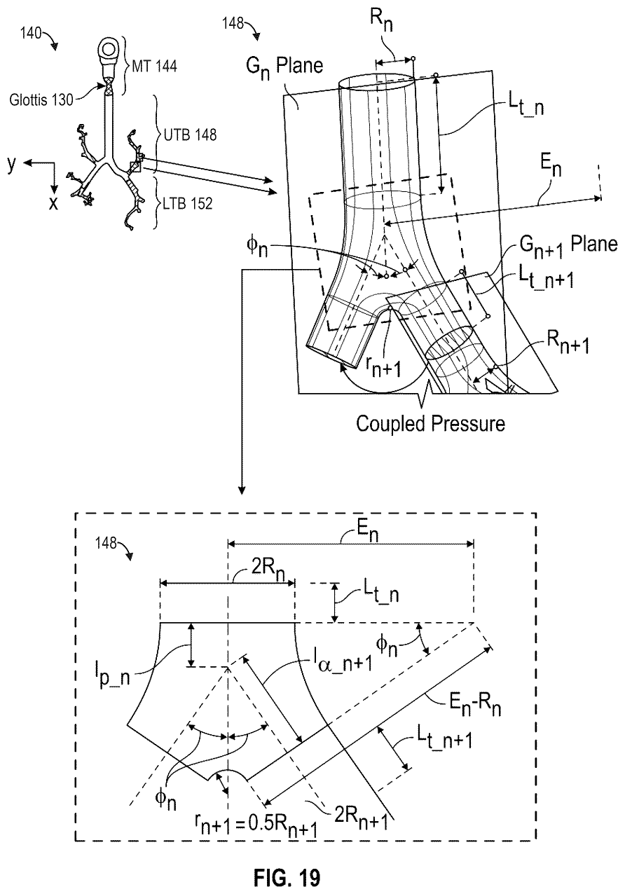

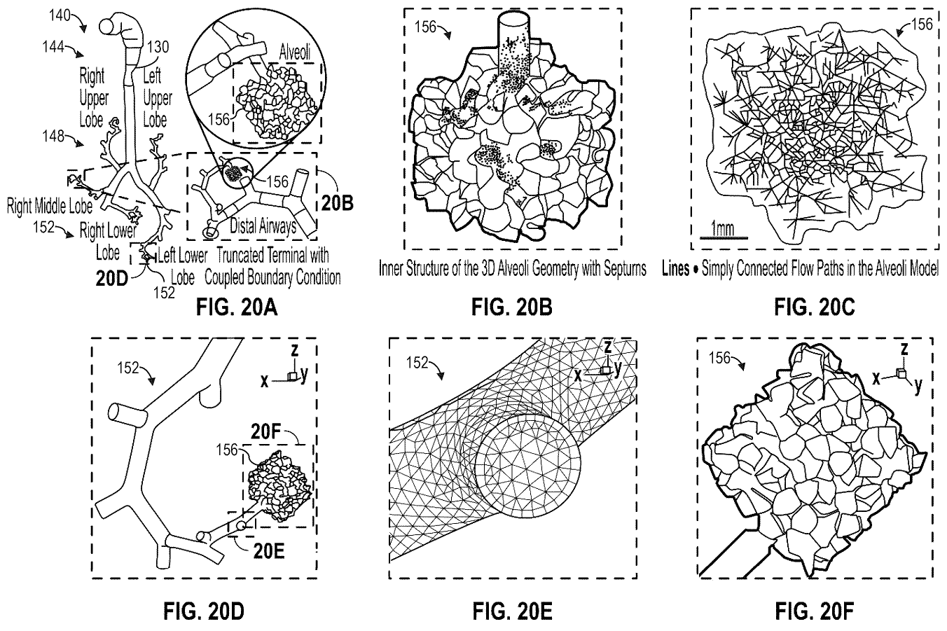

FIGS. 19 and 20A-20F illustrate another exemplary embodiment of an in situ model 140 (hereinafter the “elastic TWL model 140”) configured to reconstruct airways tree such that airways branch follows the rules of regular dichotomy after G3 to G17. Regular dichotomy means that each branch of a treelike structure gives rise to two daughter branches of identical dimensions. With such simplification, the TWL modeling strategy can be a feasible method to reduce the computational cost for the lung aerosol dynamics simulations from mouth and nose to alveoli without sacrificing computational accuracy.

The elastic TWL model 140, which is a multi-path whole-lung model, consists of four sections: (1) mouth-to-throat (MT) 144; (2) upper tracheobronchial (UTB) airways 148 extending through G1 (second bifurcations); (3) Five lower tracheobronchial (LTB) 152 airways up to G17, representing the unsymmetrical 5-lobe human pulmonary routes; and (4) the heterogeneous acinus 156 (shown in FIG. 20A). Specifically, the first three sections represent the conductive airway zone extending from the mouth to the lowest bronchioles right before the start of the alveolar region. The MT 144 and UTB 148 geometries may be created based on the realistic airway model of the upper airway constructed from the computerized tomography (CT) data of a healthy patient, for example. The LTB 152 geometry may be constructed using SolidWorks (Dassault Systemes SolidWorks Corporation, Waltham, MA), with the symmetry assumption that the branching angles (φₙ) are the same in the bifurcations at the same generation. FIG. 19 shows the schematic outline of the construction of the symmetric path model of the airway. The dimensions of the bronchi, i.e., airway radius (Rₙ), straight segment length (L_{t_n}), and branching angle (φₙ) may be based on data from the International Commission on Radiological Protection (ICRP). The radius of the carinal ridge (rₙ) may be equal to 0.5Rₙ. Each bifurcation was created in a different plane with an inclination angle (ψₙ), as indicated by the Gₙ Plane and Gₙ₊₁ Plane as shown in FIG. 19. The range of p, may be from 30 to 65 degrees, and was determined by a series of random numbers generated in the same range. It is worth mentioning that the LTB 152 geometry can be fully defined with parameters Rₙ, L_{t_n}, φₙ, rₙ, and ψₙ. Table 4 lists all the parameters used for the LTB 152 airways geometry generation.

TABLE 4
Geometric characteristics of the human respiratory tract.

| | | Straight | | Radius of | | | | | Total |
|---|---|---|---|---|---|---|---|---|---|
| | Airway | segment | Branching | carinal | Inclination | | | | branch |
| | radius | length | angle | ridge | angle | | | | length |
| Generation | Rₙ | Lₜ_{_{—}}ₙ | φₙ | rₙ | ψₙ | Eₙ | lₐ_{_{—}}ₙ | lₚ_{_{—}}ₙ | Lₙ |
| Gₙ | (mm) | (mm) | (degree) | (mm) | (degree) | (mm) | (mm) | (mm) | (mm) |
| 2 | 4.250 | 15.00 | 35 | — | — | 25.458 | — | 3.791 | 18.791 |
| 3 | 3.050 | 8.30 | 28 | 1.525 | 53 | 11.097 | 8.604 | 2.17 | 18.921 |
| 4 | 2.200 | 9.00 | 35 | 1.1 | 35.7 | 8.021 | 4.713 | 2.205 | 15.918 |
| 5 | 1.800 | 8.10 | 39 | 0.9 | 54.7 | 8.85 | 3.334 | 2.080 | 13.514 |
| 6 | 1.450 | 6.60 | 34 | 0.725 | 31.1 | 3.135 | 3.225 | 1.059 | 10.884 |
| 7 | 1.200 | 6.00 | 48 | 0.6 | 33.4 | 1.965 | 1.621 | 0.729 | 8.350 |
| 8 | 1.000 | 5.30 | 53 | 0.5 | 58.8 | 1.515 | 1.130 | 0.556 | 6.986 |
| 9 | 0.825 | 4.37 | 54 | 0.4125 | 41.1 | 1.464 | 0.899 | 0.505 | 5.774 |
| 10 | 0.675 | 3.62 | 51 | 0.3375 | 63.3 | 1.564 | 0.820 | 0.483 | 4.923 |
| 11 | 0.545 | 3.01 | 46 | 0.2725 | 31.2 | 1.19 | 0.789 | 0.374 | 4.173 |
| 12 | 0.440 | 2.50 | 47 | 0.22 | 45.4 | 1.183 | 0.615 | 0.486 | 3.602 |
| 13 | 0.410 | 2.7 | 48 | 0.205 | 43.4 | 0.545 | 0.554 | 0.126 | 2.750 |
| 14 | 0.300 | 1.70 | 52 | 0.15 | 31.6 | 0.875 | 0.352 | 0.313 | 2.365 |
| 15 | 0.265 | 1.38 | 45 | 0.1325 | 47.4 | 1.078 | 0.397 | 0.399 | 2.176 |
| 16 | 0.255 | 1.10 | 42 | 0.1275 | 32 | 0.576 | 0.425 | 0.236 | 1.761 |
| 17 | 0.230 | 0.92 | 50 | 0.115 | — | — | — | — | — |

The total branch length (Lₙ) is defined as the sum of three lengths (see Eq. (25)) (i.e., the length of a segment contained in the daughter portion of the previous bifurcation (l_{a_n}), a straight length of the generation n (L_{t_n}), and the length of a segment contained in the parent portion of the successive bifurcation (l_{p_n})). The total branch length Lₙ of the generation n (Gₙ) can be expressed as:

$${L}_{n} = {l}_{a \_ n} + {L}_{t \_ n} + {l}_{p \_ n} \qquad ( EQ . 25 )$$

$$where$$

$${l}_{p \_ n} = {E}_{n} tan {ϕ}_{n} - \frac{{E}_{n} / cos {ϕ}_{n} - ( {E}_{n} - {R}_{n} + {R}_{n + 1} )}{sin {ϕ}_{n}} \qquad ( EQ . 26 )$$

$${l}_{a \_ n} = \frac{{E}_{n - 1} ( 1 - cos {ϕ}_{n - 1} ) + ( {R}_{n - 1} - {R}_{n} )}{sin {ϕ}_{n - 1}} cos {ϕ}_{n - 1} \qquad ( EQ . 27 )$$

Based on the symmetry assumption, the geometry of the LTB 152 may be reduced by truncating one of the daughter branches of each bifurcation in the model to reduce computational cost. The airflow pressure at the truncated plane may be paired with the pressure of the cross-sectional plane at the corresponding location of the paring daughter branch.

An illustration of the acinus structure 156 and its dimensions are shown in FIGS. 20A-20F. Specifically, the average volume of the five acini 156 (i.e., one for each lobe) is 6.2e-9 m³, which is the residual volume (RV). The acinar geometry contains 406 alveoli with a mean generation of 6.7 (see Table 5).

TABLE 5
Geometric details of the heterogeneous acinus model.

| | |
|---|---|
| No. of alveoli | 406 |
| Min. generation | 3 |
| Max. generation | 11 |
| Mean generation | 6.7 |

As shown in FIGS. 20A-20F, the tetrahedral mesh with six near-wall hexahedral prism layers was generated using Ansys Fluent Meshing 2020 R2 (Ansys Inc., Canonsburg, PA). Mesh independence test was performed to find the mesh with the best balance between computational accuracy and time (see Supplementary Online Material (SOM) for more details). The mesh has 31,867,870 cells and the minimum orthogonal quality is 0.12.

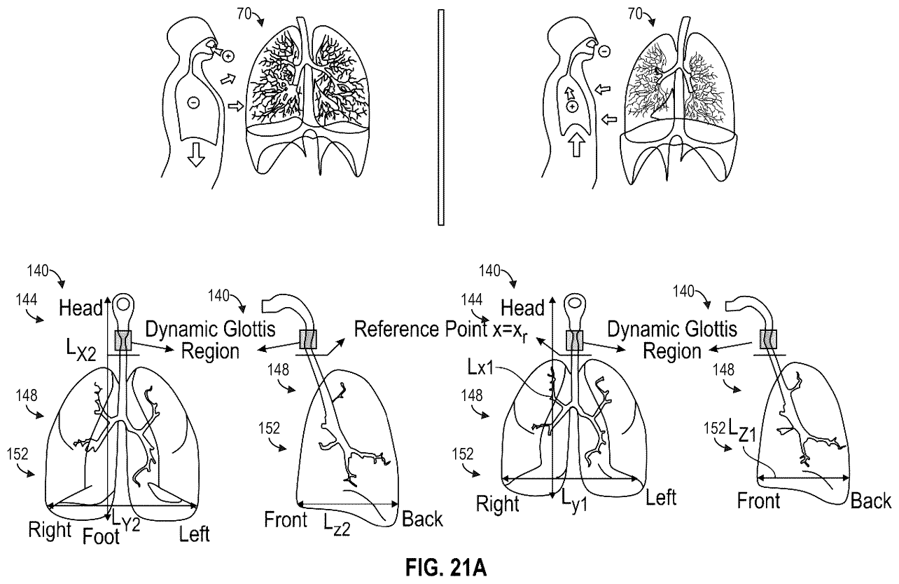

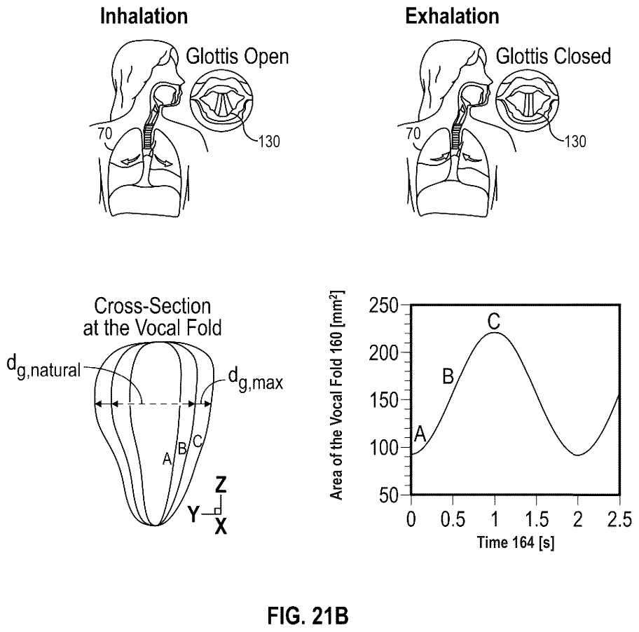

The airway deformation kinematics in a full inhalation-exhalation breathing cycle are shown in FIGS. 21A and 21B, which includes the expansion-contraction motion of the TB tree and motion of the glottis 130. Dynamic mesh method may be employed to describe the temporal and spatial nodal displacements of the computational domain, achieved using in-house C programs. The prescribed airway deformation can be defined mathematically. Specifically, the airway wall from trachea to G17 expands and contract in all three directions (i.e., head-foot (x), arm-arm (y), and back-front (z) directions) with anisotropic deformation ratio x:y:z=1:0.375:1. The reduced deformation in y direction is due to the rib cage restriction. Furthermore, the glottis region 130 opens and closes only in the y direction. To define the above-mentioned airway deformation kinematics, a generalized function to prescribe the nodal displacements of the airway walls is given by:

$${x}_{i}^{n} = {x}_{i , r} + \frac{{f}_{t} ( {t}^{n} )}{{f}_{t} ( {t}^{n - 1} )} {f}_{s} ( {x}_{i}^{n - 1} ) ( {x}_{i}^{n - 1} - {x}_{i , r} ) \qquad ( EQ . 28 )$$

$${f}_{t} ( {t}^{n} ) = 1 + \frac{{d}_{t , i}}{2} ( 1 - cos \frac{2 π {t}^{n}}{{T}_{c}} ) \qquad ( EQ . 29 )$$

$${f}_{s} ( {x}_{i}^{n} ) = \{ \begin{matrix}0.5 [ 1 − cos \frac{( {x}_{i}^{n} − {x}_{b} ) π {t}^{n − 1}}{{x}_{a} − {x}_{b}} ] , for trachea ( i = 1 ) \\ 0.5 [ 1 − cos \frac{( {x}_{i}^{n} − {x}_{a} ) π {t}^{n − 1}}{{x}_{b} − {x}_{a}} ] , for trachea ( i = 2 , 3 ) \\ 1 , for other regions\end{matrix} \qquad ( EQ . 30 )$$

wherein xᵢ=(x,y,z) is the coordinate of each node within the dynamic region (excluding glottis 130), x_{i,r}=(xᵣ,yᵣ,zᵣ) is the reference point, tⁿ is the current time step, T_{c} is the time period of a full breathing cycle, and d_{t,i} are the deformation ratios of airways. To achieve a smooth transition from the location where the expansion and contraction starts at the trachea to the first bifurcation,

$${f}_{s} ( {x}_{i}^{n} )$$

was integrated into Eq. (4).

$${f}_{s} ( {x}_{i}^{n} )$$

is defined by Eq. (6), in which xₐ and x_{b} are the x-coordinates defining the upper and lower boundaries of the smooth transition region in trachea. In one example for the elastic TWL model 140, xₐ=0.12 m and x_{b}=0.18 m, where the center of the human mouth opening 110 is located at x=0.

The glottis motion functions and corresponding numerical investigation results may be found in previous publications. Specifically, the glottis motion functions may be expressed as:

$${x}_{i}^{n} = ( {d}_{g , r} - 1 ) {f}_{g} ( {x}_{i}^{n - 1} ) g ( {t}^{n} ) + {x}_{i , g}^{0} \qquad ( EQ . 31 )$$

$${f}_{g} ( {x}_{i}^{n} ) = \{ \begin{matrix}{sin}^{m} ( \frac{{x}_{1}^{n - 1} - {x}_{g , a}}{{x}_{g , b} - {x}_{g , a}} π ) , & for i = 2 \\ 0 , & for i = 1 and 3\end{matrix} \qquad ( EQ . 32 )$$

$$g ( {t}^{n} ) = {a}_{0} + {\sum }_{β = 1}^{n} [ {a}_{β} cos ( β ω {t}^{n - 1} ) + {b}_{β} sin ( β ω {t}^{n - 1} ) ] \qquad ( EQ . 33 )$$

where

$${x}_{i , g}^{0}$$

is the initial coordinates of the node in the moving glottis region 130, and d_{g,r} is the deformation ratio of glottis 130 between maximum glottis width and the width of the glottis 130 at the neutral position. Similarly, x_{g,a}=0.056 m and x_{g,b}=0.076 m are the x coordinates that define the boundaries of smooth transition in the glottis region 130. In addition, the nodal displacement function g(tⁿ) is a time-dependent Fourier series that controls the nodal motion separately. It is worth mentioning that g(tⁿ) is simplified as a single-term sinusoidal function, which is employed to simulate the idealized glottis motion (i.e., the area of the vocal fold 160 as a function of time 164) (shown in FIG. 21B).

By adjusting the values of d_{t,i}, the elastic TWL model 140 can simulate disease-specific airway deformation kinematics representing a healthy lung and lungs with multiple COPD conditions. The values of d_{t,i} and the corresponding lung conditions are listed in Table 6.

TABLE 6
Deformation ratio of airways for different lung conditions.

| d_{t, i} | 0.4 | 0.36 | 0.2 |
|---|---|---|---|
| Lung Condition | Healthy | Mild COPD | Severe COPD |

Airflow may be assumed to be isothermal and incompressible (ρ=1.204 kg/m³), with a dynamic viscosity μ=1.825e-5 Pa·s. The continuity and Navier-Stokes (N-S) equations with moving boundaries can be given by:

$$\frac{\partial ( {u}_{i} - {u}_{i}^{mov} )}{\partial {x}_{i}} = 0 \qquad ( EQ . 34 )$$

$$\frac{\partial {u}_{i}}{\partial t} + ( {u}_{j} - {u}_{j}^{mov} ) \frac{\partial {u}_{i}}{\partial {x}_{j}} = - \frac{1}{ρ} \frac{\partial p}{\partial {x}_{i}} + \frac{μ}{ρ} \frac{\partial {τ}_{ij}}{\partial {x}_{j}} + {g}_{i} \qquad ( EQ . 35 )$$

$${τ}_{ij} = μ [ ( \frac{\partial {u}_{i}}{\partial {x}_{j}} + \frac{\partial {u}_{j}}{\partial {x}_{i}} ) - \frac{2}{3} μ {δ}_{ij} \frac{\partial {u}_{k}}{\partial {x}_{k}} ] \qquad ( EQ . 36 )$$

The convective velocity

$${u}_{i} - {u}_{i}^{mov}$$

in Eq. (35) is induced by the difference between the air velocity uᵢ and the dynamic mesh velocity

$${u}_{i}^{mov}$$

describing the airway deformation.

$${u}_{i}^{mov}$$

can be given by:

$${u}_{i}^{mov} = \partial {x}_{i} / \partial t \qquad ( EQ . 37 )$$

wherein xᵢ for the region from the trachea to alveoli (i.e., x₁>0.12 m) can be obtained from Eq. (29) and xᵢ of the moving glottis region 130 (i.e., 0.056 m<x₁<0.076 m) can be obtained from Eq. (33). The transitional characteristics of the pulmonary airflow are modeled using k-ω Shear Stress Transport (SST) model.

Particles 78 may be assumed to be spheres with constant aerodynamic diameter. The velocity and trajectory of every single particle 78 may be calculated by solving Newton's second law, which considering the drag force, gravitational force, random force induced by Brownian motion and the force induced by turbulence dispersion. Furthermore, the regional deposition of particles 78 in the airways can be calculated by RDF 94, i.e.:

$${DF}_{specific regon} = \frac{Mass of particles deposited in a specific region}{Mass of particles injected through the mouth opening} \qquad ( EQ . 38 )$$

Starting time and initial conditions of the airway model are at the end of a previous inhalation-exhalation cycle, which mimics the inhalation of aerosolized API particles 78c in real-world inhalation therapy scenarios. At the end of exhalation, the lung capacity is equal to the residual volume defined in the PFT. The pressure of the truncated branch outlet is coupled with the pressure of the identical surface at its paired daughter branch (shown in FIG. 19). A full breathing cycle of 2 seconds may be simulated, for example, including both inhalation and exhalation. The breathing profile at the mouth 110 may be determined by the lung deformation kinematics. Accordingly, for the elastic TWL model 140, the pressure-inlet boundary condition may be specified at the human mouth opening 110, where an atmosphere pressure is assumed. In one example, a total of 50,000 particles 78 are released at the mouth 110 from time t=0.2 s to 0.25 s, which is aligned with the duration of API particle 78c emissions from the inhalers 14. Specifically, 10,000 particles 78 are injected per 0.001 s. The initial velocity of particle 78 is set to 0, as the particles 78 can be accelerated to the flow velocity 127 within the extending section at the human mouth opening 110 (see FIGS. 20A-20F). Particles 78 are considered “deposited” when the distance between the center of the particle 78 and the airway wall is less than the particle radius.

The numerical approach of the elastic TWL model 140, may be based on a predetermined dynamic mesh method, one-way coupled Euler-Lagrange method, and k-ω Shear Stress Transport (SST) model, to enable predictions of anisotropic airway deformation and air-particle flows in the whole-lung in tandem where turbulent, transitional, and laminar flows coexist. To that end, UDFs may be developed and compiled for specifying the airway deformation kinematics; specifying the coupled pressure boundary conditions at truncated branch outlets; recovering the anisotropic corrections on turbulence fluctuation velocities; modeling the Brownian motion induced forces; storing particle deposition data, and the like.

The CFPD simulations may be executed using Ansys Fluent 2020 R2 (Ansys Inc., Canonsburg, PA) The Semi-Implicit method for pressure-linked equations (SIMPLE) algorithm may be employed for the pressure-velocity coupling, and the least-squares cell-based scheme may be applied to calculate the cell gradient. The second-order scheme may be employed for pressure discretization. In addition, the second-order upwind scheme may be applied for the discretization of momentum and turbulent kinetic energy. Convergence is defined for continuity, momentum, and supplementary equations when residuals are lower than 1.0e-5. Depending on the particle size simulated and the lung conditions, the computational time for completing the elastic TWL model 140 on OSU HPCC ranges may be between approximately 118 and 152 hours. The computational time for completing the static TWL model 188 on OSU HPCC ranges may be between approximately 22 and 42 hours.

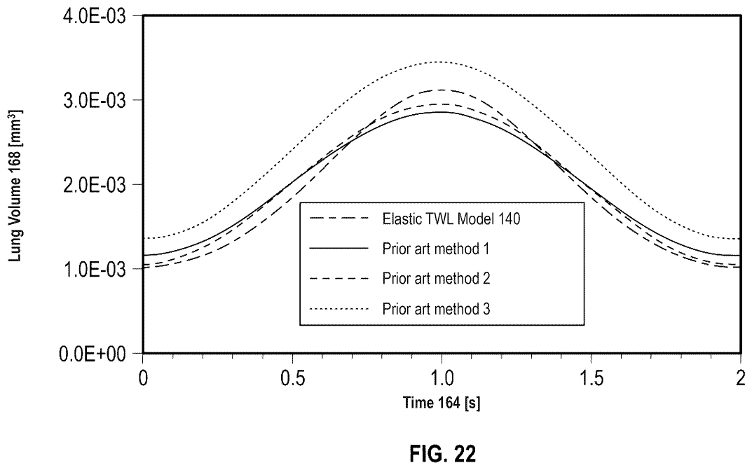

The elastic TWL model 140 may be validated by comparing the change in total lung volume 168 during a full breathing cycle predicted by the numerical method with experimentally measured results from the literature as shown in FIG. 22. It should be noted that the initial lung volume 168 equals residual volume (RV). Moreover, to calculate the whole lung volume 168 of the elastic TWL model 140, the acinus volume is multiplied by 2¹⁵ (i.e., 15 generations were truncated) to recover the total volume of a whole lung. The total lung volume 168 through breathing matches well with the data in the open literature. Thus, the generalized airway deformation function and the elastic TWL model 140 may be able to capture the deformation kinematics of a real human respiratory system.

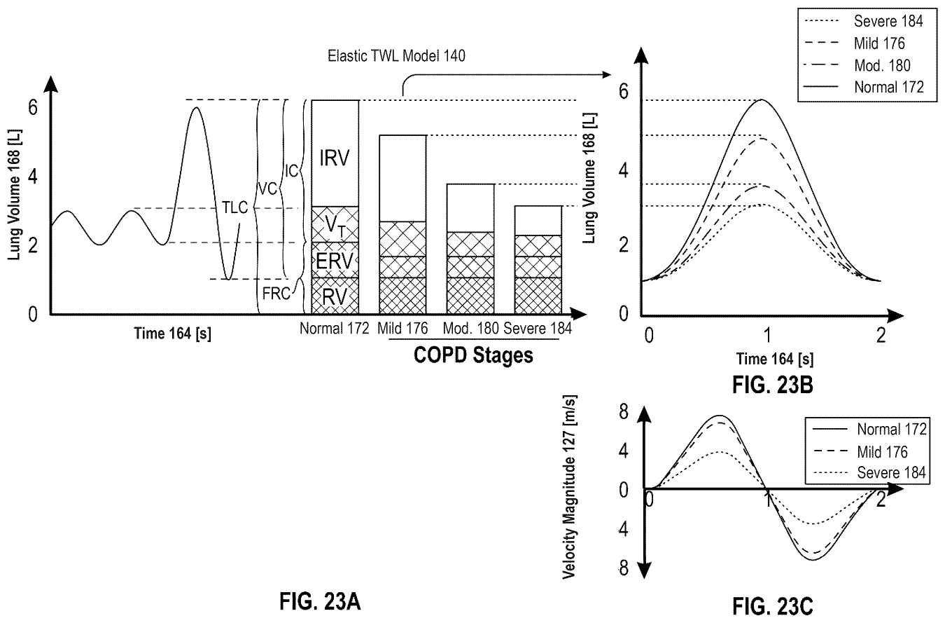

To model the disease-specific airway deformation kinematics, the elastic TWL model 140 may be further calibrated by varying the values of d_{t,i}. Specifically, the values of d_{t,i} may be determined by matching the total lung capacity (TLC) under two COPD conditions (i.e., mild and severe COPD) as well as the TLC of a healthy lung. It should be noted that lung RVs are assumed to be the same for healthy and diseased lungs. Lung volumes under different health conditions, including one healthy or “normal” condition 172 and three stages of COPD (i.e., a Stage I or “mild” COPD condition 176, a Stage 2 or “moderate” COPD condition 180, and a Stage III or “severe” COPD condition 184) are given in FIG. 23A. Correspondingly, the lung volume changes calculated using the elastic TWL model 140 are given in FIG. 23B. The value of d_{t,i} for different lung conditions is given in Table 6.

As shown in FIG. 23A, “ERV” refers to Expiratory Reserve Volume, “FRC” refers to Functional Residual Capacity, “IC” refers to Inspiratory Capacity, “IRV” refers to Inspiratory Reserve Volume, “RV” refers to Residual Volume, “TLC” refers to Total Lung Capacity, “V_{T}” refers to Tidal Volume, and “VC” refers to Vital Capacity.

The k-ω SST model may be validated and employed to resolve the flow field based on its ability to predict pressure drop, velocity profiles accurately, and shear stress for both transitional and turbulent flows. Specifically, the representative Reynolds number (Re) and TKE 128 at the peak of inhalation (t=0.5 s) in multiple generations are shown in Table 7. At the peak inhalation, the airflow is turbulence from mouth to G5 and the flow relaminarization happens after G5. Therefore, during the full inhalation-exhalation cycle, the airflow is mainly laminar-to-turbulence transitional flow in the mouth-to-G5 region, and laminar in the G5-to-alveoli region. The one-way coupled Euler-Lagrange method may also be validated using in vitro and in vivo data in previous research for accurate predictions of the aerosol dynamics in human respiratory systems.

TABLE 7
Typical Reynolds numbers (Re) and TKE 128 at different locations
of the airway at the peak inhalation (t = 0.5 s).

| | Normal Condition 172 | | Severe COPD Condition 184 | |
|---|---|---|---|---|
| | Re | TKE 128 | Re | TKE 128 |
| Oral cavity | 6.68E+3 | 2.16E−01 | 4.51E+3 | 8.72E−2 |
| Vocal folds | 1.44E+4 | 1.78E+00 | 9.90E+3 | 7.82E−01 |
| G0 | 1.07E+4 | 1.65E+00 | 7.32E+3 | 8.44E−01 |
| G2 | 4.95E+3 | 2.32E+00 | 3.40E+3 | 1.15E+00 |
| G3 | 3.74E+3 | 1.29E+00 | 2.49E+3 | 5.46E−01 |
| G5 | 1.31E+3 | 4.49E−01 | 8.34E+2 | 1.50E−01 |
| G6 | 8.97E+2 | 3.11E−01 | 5.86E+2 | 1.02E−01 |
| G7 | 5.78E+2 | 2.05E−01 | 3.81E+2 | 5.20E−2 |
| G17 | 3.53E+00 | 1.0E−14 | 1.43E+00 | 1.0E−14 |

The particle DF 94 may be predicted using a static TWL model 188 at a steady inhalation flow rate of 30 L/min compared with both numerically predicted and experimentally measured data from open literature. Table 8 compares the total DF 94 of particles 78 with dₚ=1.0, 2.0 and 5.0 μm. In general, the total DF 94 either predicted by numerical methods or measured experimentally follow the same trend as dₚ increases from 1.0 to 5.0 μm. The static TWL model 188 may predict slightly lower total DF 94 for all three sizes of particles 78 tested. This difference in total DF 94 could be related to the different airway structures.

TABLE 8
Total lung DF 94 comparison with benchmark
deposition data in previous literature.

| dₚ | Static TWL | 2016 | 1989 |
|---|---|---|---|
| [μm] | Model 188 | Benchmarks | Benchmarks |
| 1.0 | 17.5% | 32.8% | 24.2% |
| 2.0 | 38.4% | 44.2% | 45.3%. |
| 5.0 | 71.5% | 75.4% | 81.0% |

The pulmonary airflow features (i.e., laminar-to-turbulence transition and relaminarization) may be determined. Specifically, the representative Reynolds number (Re) and turbulence kinetic energy (TKE) 128 at peak inhalation at different generations in the whole-lung model are listed in Table 7. It can be noted that at peak inhalation (t=0.5 s), the airflow in the upper airway (i.e., above G5) is mainly turbulence, although the TKE 128 in the oral cavity 114 is low. The flow fluctuation increases in the glottis regions 130 with the laryngeal jet extended into G3. It can be observed from Table 7 that TKE 128 increases from G0 to G2, which can be due to the reduced hydraulic diameter. After airflow passes G5, relaminarization starts. Re decreases gradually from G5 to alveoli. Re is less than 2 at G17. In addition, healthy lung deformation kinematics resulted in higher Re and TKE 128 than severe COPD lung at all monitoring locations selected from mouth to alveoli.

To evaluate the significance of airway deformation on pulmonary airflow characteristics and determine the necessity to employ the elastic TWL model 140, the pulmonary airflow fields predicted by the static TWL model 188 and the elastic TWL model 140 may be compared. The static TWL model 188, which is widely used, has two major differences compared with the elastic TWL model 140. First, the static TWL model 188 may use velocity mouth and nose inlet conditions instead of realistic pressure boundary conditions due to the absence of the acinus structure 156 in the static TWL model 188. Second, the static TWL model 188 may neglect glottis 130 and TB tree deformation kinematics.

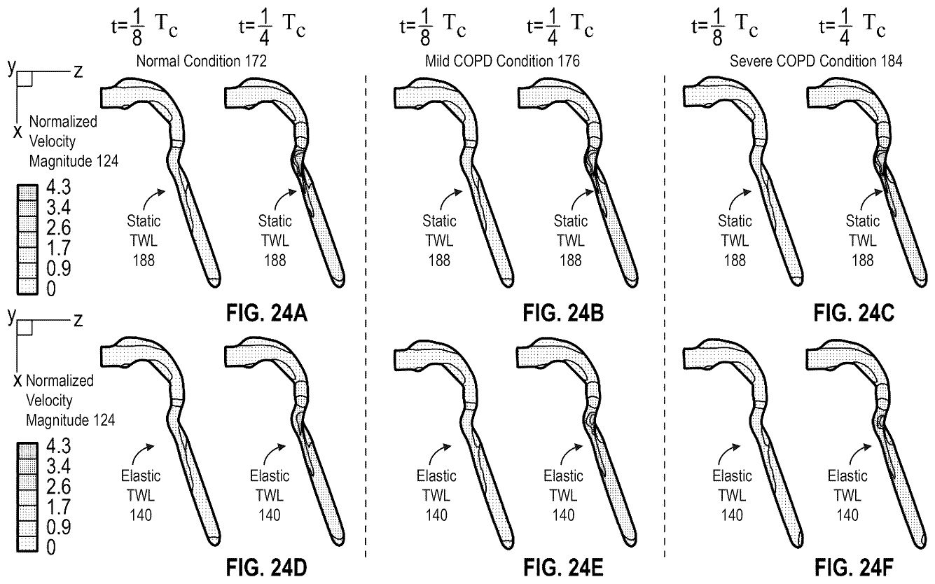

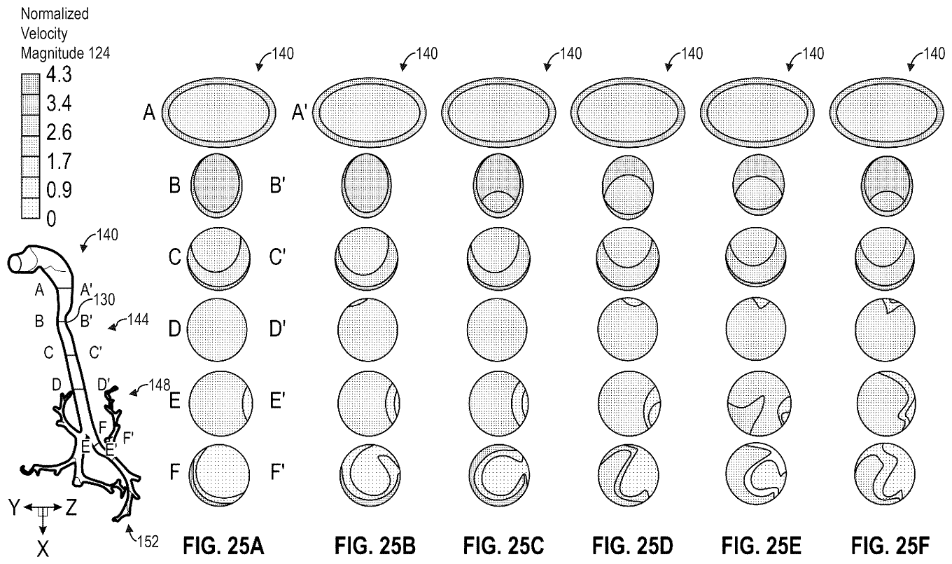

To compare the airflow fields, one full breathing cycle was simulated for three lung conditions, i.e., the normal condition 172, the mild COPD condition 176, and the severe COPD condition 184, using the elastic TWL model 140. The static TWL model 188 may also predict the airflow structure for those three lung conditions, with sinusoidal breathing mass flow rate waveforms applied at the human mouth opening 110. The sinusoidal waveform functions providing the equivalent lung volume 168 changes, which were obtained from the elastic TWL model 140 results to minimize the influence of potential boundary condition differences between the static TWL model 188 and the elastic TWL model 140. The comparisons of inspiratory airflow structures at the sagittal plane are shown in FIGS. 24A-24F and 25A-25F. The normalized velocity ∥ [symbol] ∥ is nondimensionalized using the averaged velocity at the human mouth opening 110 at the peak inhalation flow rate

$$( t = \frac{1}{4} {T}_{c} ) .$$

Since the inhaled particle transport and deposition are dominantly influenced by the inspiratory airflow, FIGS. 24A-24F show the normalized velocity contour at the sagittal plane (y=0) at

$$t = \frac{1}{8} {T}_{c} and t = \frac{1}{4} {T}_{c} .$$

The airflow pattern during inhalation changes significantly as the flow rate reaches its peak value. The mouth jet and laryngeal jet become much stronger at

$$t = \frac{1}{4} {T}_{c}$$

than

$$t = \frac{1}{8} {T}_{c} .$$

All six cases show similar inspiratory airflow structure, except that the elastic TWL model 140 predicts relatively weaker laryngeal jets extended from the glottis 130 than the static TWL model 188 for all three lung conditions. Such differences may be due to the wider glottis openings in the elastic TWL model 140 than the static TWL model 188. In addition, the elastic TWL model 140 predicts weaker convection in the oropharynx 118 for severe COPD conditions compared with normal and mild COPD conditions, which is due to the decreases in TB tree expansion amplitude with the increase in the COPD severity.

To further visualize the lung deformation effect on airflow patterns in MT 144, trachea, and G1-to-G3 regions, ∥ [symbol] ∥ contours and tangential velocity vector distributions on selected cross-sections (i.e., AA′ to EE′) at the peak inhalation flow rate

$$( t = \frac{1}{4} {T}_{c} )$$

are given in FIGS. 25A-25F. Specifically, the flow structures shown in AA′ are similar for all six cases, with no evident differences in secondary flows. This indicates that during the inhalation, the glottis motion and TB expansion have minor effect on the airflow patterns in the oropharynx 118 since viscous dissipation effect on the airflow patterns. At BB′ where is the glottis 130, one can notice the glottis expansion in elastic TWL model 140 cases. As a result of the glottis expansion, differences in airflow patterns can be observed at BB′ between the static TWL model 188 and the elastic TWL model 140 simulation results. For normal conditions, although both the static TWL model 188 and the elastic TWL model 140 simulations predict counterclockwise in-plane recirculation near the center of BB′, the vortices locate more to the left in the elastic TWL model 140 than the static TWL model 188. Also, the secondary flow has different directions on the top left corner of BB′. In addition, ∥ [symbol] ∥ at CC′ and DD′ shows the skewed velocity distributions induced by the laryngeal jets in the trachea. It can be seen from CC′, two counter-rotating vortices are formed at the center of CC′ in the static TWL model 188, while only one counterclockwise vortex can be observed in the elastic TWL model 140. The reason for such differences is determined by whether the glottis 130 and trachea expansion are included or neglected in the TWL model. Explicitly, the vocal fold and trachea expand during inhalation. Thus, compared with the elastic TWL model 140, the static TWL model 188 predicts higher flow velocity 127 at the throat-to-trachea region and higher intensity of laryngeal jet impact, hence possibly higher shear velocity, which leads to two vortices at CC′. In contrast, only one counterclockwise vortex is preserved at CC′ in the elastic TWL model 140 due to the larger cross-sectional area induced weaker secondary flow intensities. Moreover, ∥ [symbol] ∥ contour at CC′ shows that the static TWL model 188 predicts higher ∥ [symbol] ∥ at the anterior of the trachea (i.e., bottom of CC′) for the normal condition 172 and the mild COPD condition 176 than the other conditions. In slice DD′, the counterclockwise secondary flow existing upstream is diminished and challenging to be observed. As the flow enters the first bifurcation (i.e., EE′), airflow structures between the static TWL model 188 and the elastic TWL model 140 are highly different. For the static TWL model 188, vortices can be found on both left and right sides in EE′. However, in the elastic TWL model 140, the vortices shift to the top-right and bottom left of slice EE′. After the third bifurcation (i.e., FF′), the airflow structure is affected by lung deformation kinematics and the inhalation flow rate (lung conditions). Specifically, at FF′, although Dean's flows can be observed in all cases, the predicted location and number of the vortices differs between the static TWL model 188 and the elastic TWL model 140. Thus, it can be concluded that the neglected airway deformation kinematics has a minor influence on the inspiratory airflow fields from mouth to AA′. In contrast, the effect of lung deformation kinematics on airflow structure becomes manifest from BB′ to FF′, which represents the glottis 130 to G3. Furthermore, it can also be concluded that the lung disease condition induced difference in airway deformation kinematics can lead to different pulmonary airflow patterns from the glottis 130 to G3 and possibly further downstream. This indicates the necessity to model airway motions on a disease-specific level.

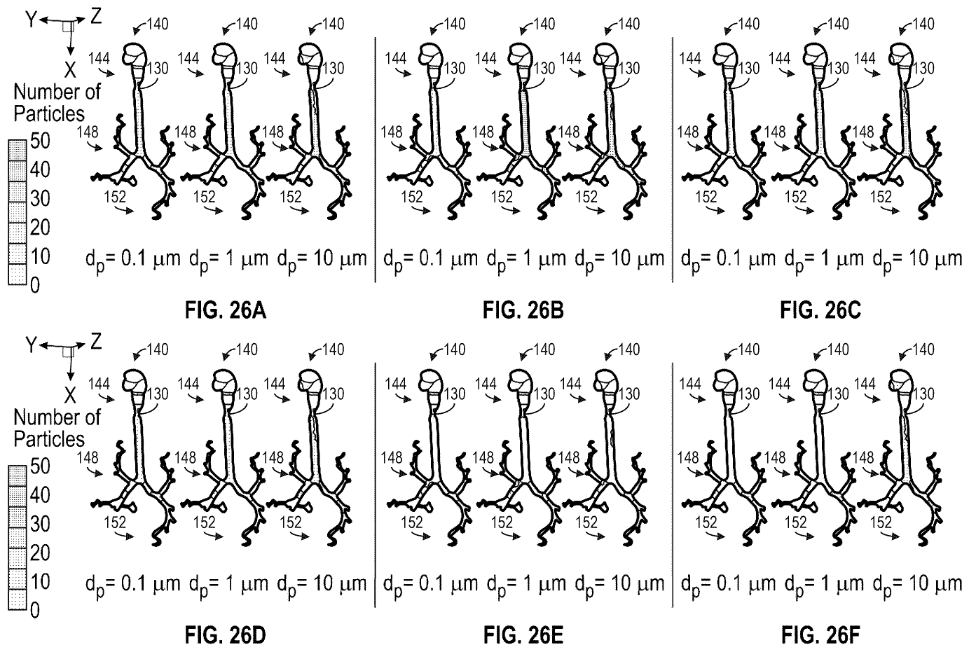

To further investigate how the neglected airway deformation kinematics can influence the predictions of lung aerosol dynamics, the transport and deposition of particles with different diameters (i.e., dₚ=0.1, 0.2, 0.5, 1.0, 2.0, 5.0 and 10.0 μm) in the static TWL model 188 and the elastic TWL model 140 are investigated individually under the above-mentioned three lung conditions. As an example, deposition patterns of particles 78 with dₚ=0.1, 1.0, and 10.0 μm in both the static TWL model 188 and the elastic TWL model 140 after one full inhalation-exhalation breathing cycle are visualized in FIGS. 26A-26F. The concentrated particle depositions occur in the throat, the main bronchus, and the first three bifurcations. However, the differences in particle delivery deposition patterns predicted by the static TWL model 188 and the elastic TWL model 140 may be significant. Specifically, at the normal lung condition, particles 78 are more likely to be entrapped in the trachea of the static TWL model 188 compared with the elastic TWL model 140. Previous research demonstrates that Brownian motion induced force has a strong impact on the transport and deposition of small particles 78 (dₚ<0.5 μm), while the inertia impaction on small particle depositions (e.g., dₚ<0.5 μm) is negligible. This explains the deposition of 0.1-μm particles 78 in the trachea for the static TWL model 188. In contrast, with the trachea expansion during the inhalation, 0.1-μm particles 78 had less chance to touch the airway wall in the elastic TWL model 140 compared with the static TWL model 188. Additionally, the static TWL model 188 also predicted a significantly higher deposition in the trachea for 1.0 μm particles 78 than the elastic TWL model 140. The deposition differences in the trachea between the static TWL model 188 and the elastic TWL model 140 are also partially due to the different intensities of the secondary flow observed in FIG. 25A at BB′ and CC′. Specifically, in the elastic TWL model 140, the wider glottis opening during inhalation induced weaker laryngeal jet impaction in the trachea, which create the difference in airflow patterns in the trachea and contribute to the deposition differences between the static TWL model 188 and the elastic TWL model 140. For the deposition patterns of 10-μm particles 78 shown in FIGS. 26A and 26D, another observation is the “delayed” particle deposition in the elastic TWL model 140 than the static TWL model 188. Specifically, although a lower deposition concentration of 10.0 μm particles 78 in the trachea is observed in the elastic TWL model 140 than the static TWL model 188, the deposition concentration is higher in the first two bifurcations of right lobes in the elastic TWL model 140. This may be due to the TB airway wall expansion reduce the chances for particles 78 to touch the airway wall, and delays the deposition of particles 78 more to the downstream airways. The static TWL model 188 predicts much higher deposition concentration in MT 144 of large particles 78 (dₚ=10 μm) than elastic TWL model 140.

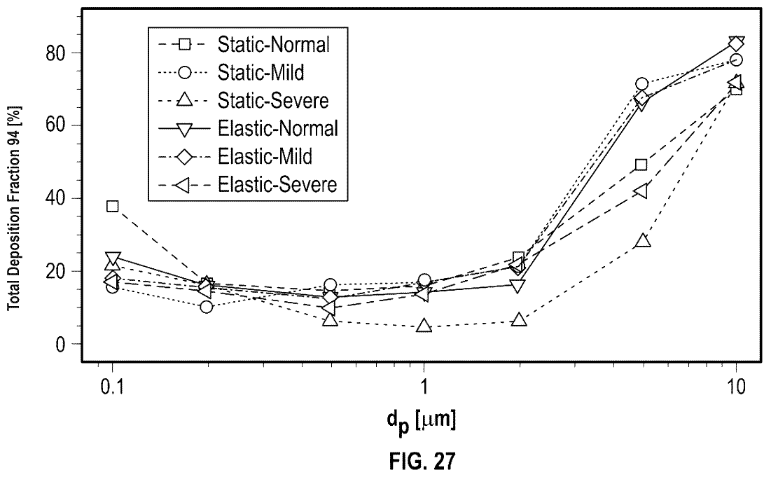

The effect of lung deformation on particle deposition may also be analyzed by comparing the total DFs 94 of particles 78 with dₚ ranging from 0.1 to 10 μm under different lung health conditions as shown in FIG. 27. In general, both the static TWL model 188 and the elastic TWL model 140 may be able to predict the classic “U-curve” total DF 94 as a function of dₚ. For lungs under normal condition 172, the static TWL model 188 predicts 13.4% higher total DF 94 of particles 78 with dₚ=0.1 μm than the elastic TWL model 140. For particle size ranging from 0.2 to 2.0 μm, the differences in total DF 94 predicted by the static TWL model 188 and the elastic TWL model 140 are relatively small which are approximately 7%. However, as particle size increases to 5.0 and 10.0 μm, the static TWL model 188 predicts 16.9% and 13.1% less total DFs 94 than the elastic TWL model 140, respectively. For the mild COPD condition 176, the difference in total DF 94 predicted by the static TWL model 188 and the elastic TWL model 140 is not obvious. Specifically, the highest difference is 5.1%, as the elastic TWL model 140 generates a higher total DF 94 for particles 78 with dₚ=0.2 μm than the static TWL model 188. For the severe COPD condition 184, both the static TWL model 188 and the elastic TWL model 140 predict similar total DF 94 for small (dₚ=0.1 and 0.2 μm) and large (dₚ=10 μm) particles 78. However, for particles 78 with dₚ between 0.5 and 5 μm, the static TWL model 188 gives lower total DFs 94 than the elastic TWL model 140. Especially for dₚ=2 μm, the static TWL model 188 predicts 16% lower total DF 94 than the elastic TWL model 140. It can be concluded that the static TWL model 188 can be used instead of the elastic TWL model 140, which is more physiologically realistic, for predicting the total DF 94 of particles 78 (0.1<dₚ<10 μm) for airways under the mild COPD condition 176 only. For other lung health conditions, the more physiologically realistic TWL model should be employed to more accurately reflect the airway deformation effect on particle transport and deposition.

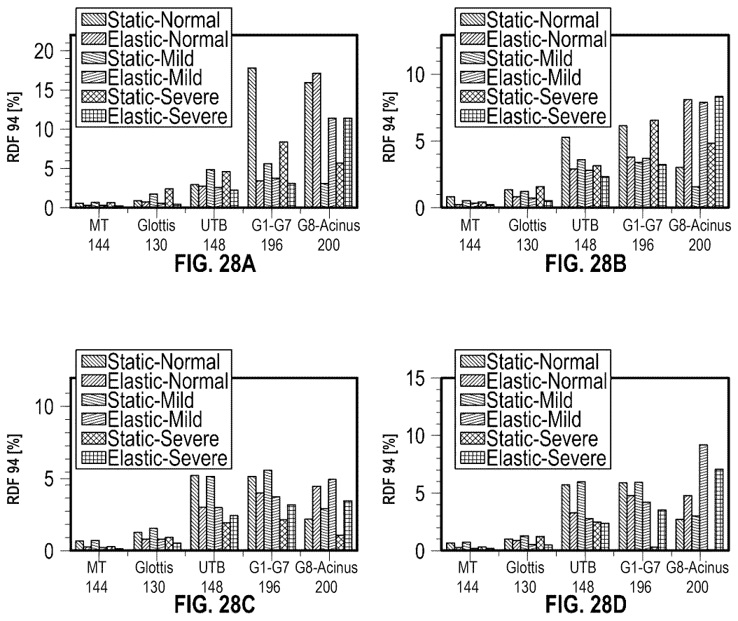

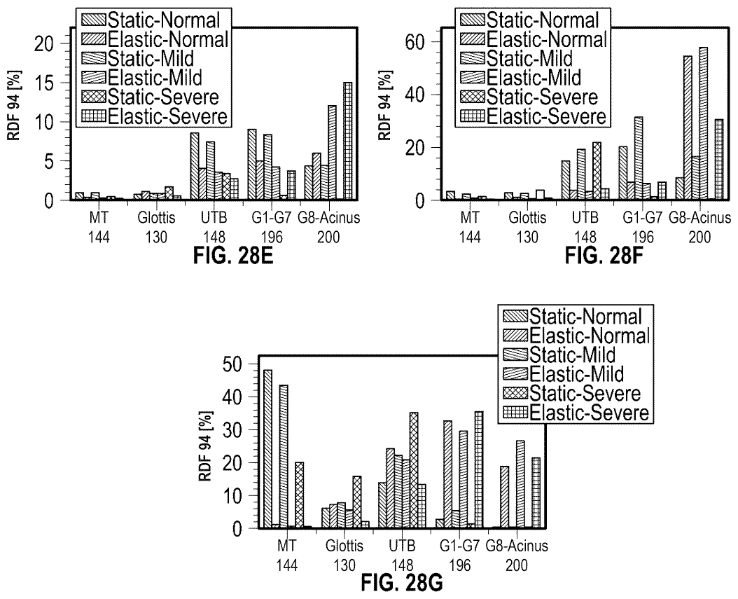

RDFs 94 predicted by the static TWL model 188 and the elastic TWL model 140 may be visualized and compared as shown in FIGS. 28A-28G. Explicitly, for particles 78 with 0.1 μm≤dₚ≤5 μm, regardless of the lung conditions (i.e., the normal condition 172, the mild COPD condition 176, or the severe COPD condition 184), the static TWL model 188 predicts higher RDFs 94 in the TB tree (from MT 144 to G7) while lower RDFs 94 in lower airways (G8 to acinus 156) than the elastic TWL model 140. The higher RDF predictions using the static TWL model 188 is due to the neglected airway expansions during the inhalation. The expansions of glottis opening and the TB tree in the elastic TWL model 140 can reduce the chance for particles 78 to touch the airway wall, with the reduced intensity of the laryngeal jet impact in the trachea thereby reducing the deposition due to the direct impaction and the afterward splash induced dispersion, especially for small particles 78 (dₚ=0.1 μm). However, with the static airway, the Brownian motion effect increases the deposition possibility for small particles. This also explains the overprediction of the static TWL model 188 on total DF 94 of particles 78 with dₚ=0.1 μm. In contrast, the lower RDF predictions from G8 to acinus 156 using the static TWL model 188 can be also due to the reduced particle interceptions in small airways resulted from the reduced secondary airflow intensities because of the negligence of the airway deformation. Specifically, interception is the dominant mechanism for particle depositions in small airways. Physiologically realistic airway deformations can enhance the localized secondary flows and thereby increasing the particle interceptions with the airway wall in the elastic TWL model 140 than the static TWL model 188.

For particles with dₚ=10 μm, inertial impaction and gravitational sedimentations may dominate transport and deposition in the airways. Similar to smaller particles 78, the simulation results show that the static TWL model 188 predicts higher RDFs 94 of 10-μm particles 78 in the upper airway (i.e., MT 144 and glottis 130) than the elastic TWL model 140. Especially in MT 144, the static TWL model 188 for healthy lung condition predicts DF_{MT} 94=47.8% in contrast to DF_{MT}94=1.8% predicted by the elastic TWL model 140. The difference indicates that the effects of the reduced secondary flow and laryngeal jet impact induced by the glottis expansion decreases 10-μm particles deposition in MT 144 and glottis 130. Furthermore, the RDFs 94 in UTB 148 and lower airways predicted by the static TWL model 188 is much lower than the elastic TWL model 140. For the static TWL model 188, most 10-μm particles 78 deposited due to inertial impaction before reaching the main bronchi, and the rest of the particles 78 either suspended in the airway or exhaled. For the elastic TWL model 140, as 10-μm particles 78 entering a G1-G7 region 196 and a G8-acinus region 200, both inertial impaction and airway deformation induced secondary flow increase the chance of particle interceptions with the airways, which leads to higher DF 94 in the G1-G7 region 196 and the G8-acinus region 200 compared with the static TWL model 188. In addition, the static TWL model 188 predicts no deposition of large particles 78 (d_{P}=10 μm) after G8, while the elastic TWL model 140 shows that the DF 94 of the particles 78 is about 18.6% for the normal condition 172. To that end, the static TWL model 188 may overpredict the DF 94 in the upper airway (i.e., from MT 144 to UTB 148) and the G1-G7 region 196, and underpredict the DF 94 in lower airways (i.e., the G8-acinus region 200) for particles 78 with 0.1 μm≤dₚ≤5 μm than the elastic TWL model 140. For large particles 78 (d_{P}=10 μm), the only difference is that the static TWL model 188 also underpredicts the DF 94 in the G1-G7 region 196. As such, to accurately evaluate the targeted API delivery efficiency 86 of inhaled API particles 78c, airway deformation kinematics may be considered in the simulations.

Using RDF 94, the differences in total DF 94 predicted by the static TWL model 188 and the elastic TWL model 140 for different lung conditions may be determined. For example, although the difference in total DF 94 between the static TWL model 188 and the elastic TWL model 140 is negligible in the mild COPD condition 176, noticeable differences may exist between the RDFs 94 predicted the static TWL model 188 and the elastic TWL model 140. Specifically, for the mild COPD condition 176, the static TWL model 188 predicted higher DF_{MT}-G₇ 94 for particles 78 with 0.1 μm≤dₚ≤5 μm. However, the higher DF_{MT}-G₇ 94 may be balanced by lower DF_{G8-acinus} 94. For the severe COPD condition 184, since the same deformation kinematics was prescribed for the conducting airways (i.e., trachea to G17), the effect of secondary flow induced by airway deformation on the particle interceptions with airway wall may be stronger than the effect in the mild COPD condition 176 (i.e., a higher flowrate compared to the severe COPD condition 184). The higher intensity of secondary flow in the TB tree leads to higher RDF 94 in both the G1-G7 region 196 and the G8-acinus region 200 in the elastic TWL model 140 under the severe COPD condition 184 than the static TWL model 188. Thus, the balance existed in total DF 94 between the static TWL model 188 and the elastic TWL model 140 for the mild COPD condition 176 may be broken under the severe COPD condition 184, as the elastic TWL model 140 predicts higher total DF 94 than the static TWL model 188 for particles 78 with 0.1 μm≤dₚ≤5 μm. For the normal condition 172, the difference in total DF 94 is obvious for small particles 78 (d_{P}=0.1 μm) and large particles 78 (d_{P}=5 μm and 10 μm). Specifically, for the normal condition 172, the static TWL model 188 predicts higher total DF 94 for small particles 78 (d_{P}=0.1 μm) compared with elastic TWL model 140 mainly because of the Brownian motion effect in the G1-G7 region 196, while the Brownian motion induced deposition is reduced in the elastic TWL model 140 due to airway expansion. For large particles 78 (d_{P}=5 μm and 10 μm), the prediction may be much higher DF_{G8-acinus} 94 resulting from the inertia and higher intensity due to the airway deformation induced secondary flow compared with the static TWL model 188, leading to the higher total DF 94 in the elastic TWL model 140 for the normal condition 172 than the static TWL model 188.

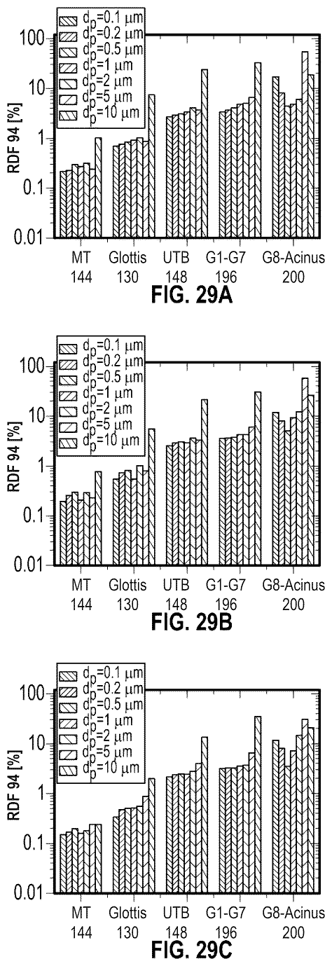

To enhance the delivery dosage of the drugs to the designated lung sites and the treatment effectiveness, the effect of disease-specific airway deformation on RDF 94 may be predicted using the elastic TWL model 140 shown in FIGS. 29A-29C, with the focus on the DF 94 in the G8-acinus region 200 (DF_{G8-acinus} 94). For example, all three lung conditions, the DFs 94 of particles 78 with 0.1≤d≤10 μm in MT 144 are less than 1%. Moreover, particles 78 with dₚ=5 μm has the highest DF_{G8-acinus} 94. With the increase in particle size, the DF_{G8-acinus} 94 first decreases (until dₚ=0.5 μm) and then increases (until dₚ=5 μm). In addition, DF_{G8-acinus} 94 of 5 μm particles 78 is higher than the DF_{G8-acinus} 94 of 10 μm particles 78. A similar DF_{G8-acinus} 94 vs. dₚ trend was predicted in previous research investigating the deep lung simulation. For the normal condition 172, DF_{G8-acinus} 94 of particles 78 with dₚ=0.1 μm is 17.1%. For particle size in 0.2≤dₚ≤2 μm, the DF_{G8-acinus} 94 is approximately 6%. However, DF_{G8-acinus} 94 increases dramatically to 54.6% for particles 78 with dₚ=5 μm. A similar trend can be observed for the mild COPD condition 176 and the severe COPD condition 184, although for the severe COPD condition 184, the highest DF_{G8-acinus} 94 is only 30.4% (when dₚ=5 μm). As such, with the exacerbation in COPD disease condition (i.e., from the normal condition 172 to the severe COPD condition 184), the highest API delivery efficiency 86 of the inhaled API particles 78c decreases indicating that delivering aerosolized medications to small airways to treat COPD may be more challenging for patients with severe disease condition. Such a phenomenon is due to the lack of airway expansion and contraction capability, which results the additional difficulty to draw the inhaled particles into the deeper airway region. Considering that better treatment for COPD can be achieved as higher drug dosage is delivered into deep airways (after G8), both small (e.g., dₚ=0.1 μm) and large particles 78 (e.g., dₚ=5 and 10 μm) are favored.

Using the elastic TWL model 140, airway deformation may be determined including airflow structure in the respiratory system from the glottis 130 to the trachea for lung conditions including, but not limited to COPD. Further, by increasing particle size from 0.1 to 10 μm, both the static TWL model 188 and the elastic TWL model 140 may predict parabolic curves for total DF 94. However, the RDFs 94 predicted by the static TWL model 188 and the elastic TWL model 140 are different as higher DF 94 (particle size in 0.1 μm≤dₚ≤10 μm) in lower airways is observed in the results from the elastic TWL model 140. With the exacerbation in COPD disease condition, the highest API delivery efficiency 86 of the inhaled API particles 78c decreases which indicates that delivering aerosolized medications to small airways to treat COPD is more challenging for patients with the severe COPD condition 184. As such, optimal size for an API particle 78c may be determined using the elastic TWL model 140 for one or more lung conditions. For example, based on the elastic TWL model 140, dₚ=5 μm is recommended as the optimal size of API particle 78c for all three lung conditions described herein (i.e., gives the highest DF_{G8-acinus} 94 based on the elastic TWL model 140 results).

Disease-specific airway deformation kinematics can significantly influence the predictions of pulmonary air-particle flow dynamics as described in further detail herein. Modeling airway deformation simultaneously with the tracking of particle-laden airflows in patient respiratory systems 70 on a disease-specific level may predict the API delivery efficiency 86 to designated lung sites or assess the occupational exposure health risks based on the lung dosimetry of the inhaled toxicants.

### ILLUSTRATIVE EMBODIMENTS

The following is a number list of non-limiting illustrative embodiments of the inventive concept disclosed herein:

1. A non-transitory computer readable medium storing a set of computer readable instructions that when executed by a processor cause the processor to:
determine a model of airway deformation in a patient-specific respiratory system using an elastic truncated whole-lung (TWL) model, the model of airway deformation having at least one designated lung site;
determine a plurality of particle airflows in the patient respiratory system for at least one disease specific level; and,
determine drug delivery efficiency to the designated lung site using the model of airway deformation and the plurality of particle airflows in the patient respiratory system.

2. The non-transitory computer readable medium of illustrative embodiment 1, wherein the set of computer readable instructions further cause the processor to determine adhesion resulting from short-range surface force of agglomeration in the patient respiratory system using the TWL model.

3. The non-transitory computer readable medium of any one of illustrative embodiments 1-2, wherein the set of computer readable instructions further cause the processor to determine carrier-API interactions in dry powder inhalers using the TWL model.

4. The non-transitory computer readable medium of any one of illustrative embodiments 1-3, wherein the set of computer readable instructions further cause the processor to determine effect of lactose carrier shape on drug delivery efficiency using the TWL model.

5. The non-transitory computer readable medium of any one of illustrative embodiments 1-4, wherein the set of computer readable instructions further cause the processor to determine effect of dry powder inhaler flow channel design on drug delivery efficiency using the TWL model.

6. The non-transitory computer readable medium of any one of illustrative embodiments 1-5, wherein the set of computer readable instructions further cause the processor to determine drug delivery deposition patterns within the patient respiratory system using the TWL model.

7. A non-transitory computer readable medium storing a set of computer readable instructions that when executed by a processor cause the processor to:
generate a one-way coupled Computational Fluid Dynamics (CFD) with Discrete Element Method (DEM) virtual whole-lung model of a patient respiratory system using Hertz-Mindlin (H-M) Johnson-Kendall-Roberts (JKR) cohesion model (CFD-DEM virtual whole-lung model), the CFD-DEM virtual whole-lung model configured to predict particle agglomeration and deagglomeration with resultant emitted aerodynamic particle size distributions (APSDs);
calibrate the CFD-DEM virtual whole-lung model;
validate the CFD-DEM virtual whole-lung model; and,
determine drug delivery efficiency and deposition patterns of a dry powder inhaler within the patient respiratory system using the CFD-DEM virtual whole-lung model.

8. The non-transitory computer readable medium of illustrative embodiment 7, wherein the set of computer readable instructions further cause the processor to determine adhesion resulting from short-range surface force of agglomeration in the patient respiratory system using the CFD-DEM virtual whole-lung model.

9. The non-transitory computer readable medium of any one of illustrative embodiments 7-8, wherein the set of computer readable instructions further cause the processor to determine carrier-API interactions in dry powder inhalers using the CFD-DEM virtual whole-lung model.

10. The non-transitory computer readable medium of any one of illustrative embodiments 7-9, wherein the set of computer readable instructions further cause the processor to determine effect of lactose carrier shape on drug delivery efficiency using the CFD-DEM virtual whole-lung model.

11. The non-transitory computer readable medium of any one of illustrative embodiments 7-10, wherein the set of computer readable instructions further cause the processor to determine effect of dry powder inhaler flow channel design on drug delivery efficiency using the CFD-DEM virtual whole-lung model.

12. The non-transitory computer readable medium of any one of illustrative embodiments 7-11, wherein the set of computer readable instructions further cause the processor to determine drug delivery deposition patterns within the patient respiratory system using the CFD-DEM virtual whole-lung model.

13. The non-transitory computer readable medium of any one of illustrative embodiments 7-12, wherein the CFD-DEM virtual whole-lung model includes a pulmonary route from mouth and nose to alveoli.

14. A method, comprising:
generating, by one or more processor, a one-way coupled Computational Fluid Dynamics (CFD) with Discrete Element Method (DEM) virtual whole-lung model of a patient respiratory system using Hertz-Mindlin (H-M) Johnson-Kendall-Roberts (JKR) cohesion model (CFD-DEM virtual whole-lung model), the CFD-DEM virtual whole-lung model configured to predict particle agglomeration and deagglomeration with resultant emitted aerodynamic particle size distributions (APSDs);
calibrating, by the one or more processor, the CFD-DEM virtual whole-lung model;
validating, by the one or more processor, the CFD-DEM virtual whole-lung model; and,
determining, by the one or more processor, drug delivery efficiency and deposition patterns of a dry powder inhaler within the patient respiratory system using the CFD-DEM virtual whole-lung model.

15. The method of illustrative embodiment 14, further comprising determining, by the one or more processor, adhesion resulting from short-range surface force of agglomeration in the patient respiratory system using the CFD-DEM virtual whole-lung model.

16. The method of any one of illustrative embodiments 14-15, further comprising determining, by the one or more processor, carrier-API interactions in dry powder inhalers using the CFD-DEM virtual whole-lung model.

17. The method of any one of illustrative embodiments 14-16, further comprising determining, by the one or more processor, effect of lactose carrier shape on drug delivery efficiency using the CFD-DEM virtual whole-lung model.

18. The method of any one of illustrative embodiments 14-17, further comprising determining, by the one or more processor, effect of dry powder inhaler flow channel design on drug delivery efficiency using the CFD-DEM virtual whole-lung model.

19. The method of any one of illustrative embodiments 14-18, further comprising determining, by the one or more processor, drug delivery deposition patterns within the patient respiratory system using the CFD-DEM virtual whole-lung model.

20. The method of any one of illustrative embodiments 14-19, wherein the CFD-DEM virtual whole-lung model includes a pulmonary route from mouth and nose to alveoli, and the step of generating the CFD-DEM virtual whole-lung model is further defined as generating, by the one or more processor, the CFD-DEM virtual whole-lung model including the pulmonary route from mouth and nose to alveoli.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set.

No element, act, or instruction used in the present application should be construed as critical or essential to the invention unless explicitly described as such outside of the preferred embodiment. Further, the phrase “based on” is intended to mean “based, at least in part, on” unless explicitly stated otherwise.

What is claimed is:

1. A non-transitory computer readable medium storing a set of computer readable instructions that when executed by a processor cause the processor to:
determine a model of airway deformation in a patient-specific respiratory system using an elastic truncated whole-lung (TWL) model, the model of airway deformation having at least one designated lung site;
determine a plurality of particle airflows in the patient respiratory system for at least one disease specific level; and,
determine drug delivery efficiency to the designated lung site using the model of airway deformation and the plurality of particle airflows in the patient respiratory system.

2. The non-transitory computer readable medium of claim 1, wherein the set of computer readable instructions further cause the processor to determine adhesion resulting from short-range surface force of agglomeration in the patient respiratory system using the TWL model.

3. The non-transitory computer readable medium of claim 1, wherein the set of computer readable instructions further cause the processor to determine carrier-API interactions in dry powder inhalers using the TWL model.

4. The non-transitory computer readable medium of claim 1, wherein the set of computer readable instructions further cause the processor to determine effect of lactose carrier shape on drug delivery efficiency using the TWL model.

5. The non-transitory computer readable medium of claim 1, wherein the set of computer readable instructions further cause the processor to determine effect of dry powder inhaler flow channel design on drug delivery efficiency using the TWL model.

6. The non-transitory computer readable medium of claim 1, wherein the set of computer readable instructions further cause the processor to determine drug delivery deposition patterns within the patient respiratory system using the TWL model.

7. A non-transitory computer readable medium storing a set of computer readable instructions that when executed by a processor cause the processor to:
generate a one-way coupled Computational Fluid Dynamics (CFD) with Discrete Element Method (DEM) virtual whole-lung model of a patient respiratory system using Hertz-Mindlin (H-M) Johnson-Kendall-Roberts (JKR) cohesion model (CFD-DEM virtual whole-lung model), the CFD-DEM virtual whole-lung model configured to predict particle agglomeration and deagglomeration with resultant emitted aerodynamic particle size distributions (APSDs);
calibrate the CFD-DEM virtual whole-lung model;
validate the CFD-DEM virtual whole-lung model; and,
determine drug delivery efficiency and deposition patterns of a dry powder inhaler within the patient respiratory system using the CFD-DEM virtual whole-lung model.

8. The non-transitory computer readable medium of claim 7, wherein the set of computer readable instructions further cause the processor to determine adhesion resulting from short-range surface force of agglomeration in the patient respiratory system using the CFD-DEM virtual whole-lung model.

9. The non-transitory computer readable medium of claim 7, wherein the set of computer readable instructions further cause the processor to determine carrier-API interactions in dry powder inhalers using the CFD-DEM virtual whole-lung model.

10. The non-transitory computer readable medium of claim 7, wherein the set of computer readable instructions further cause the processor to determine effect of lactose carrier shape on drug delivery efficiency using the CFD-DEM virtual whole-lung model.

11. The non-transitory computer readable medium of claim 7, wherein the set of computer readable instructions further cause the processor to determine effect of dry powder inhaler flow channel design on drug delivery efficiency using the CFD-DEM virtual whole-lung model.

12. The non-transitory computer readable medium of claim 7, wherein the set of computer readable instructions further cause the processor to determine drug delivery deposition patterns within the patient respiratory system using the CFD-DEM virtual whole-lung model.

13. The non-transitory computer readable medium of claim 7, wherein the CFD-DEM virtual whole-lung model includes a pulmonary route from mouth and nose to alveoli.

14. A method, comprising:
generating, by one or more processor, a one-way coupled Computational Fluid Dynamics (CFD) with Discrete Element Method (DEM) virtual whole-lung model of a patient respiratory system using Hertz-Mindlin (H-M) Johnson-Kendall-Roberts (JKR) cohesion model (CFD-DEM virtual whole-lung model), the CFD-DEM virtual whole-lung model configured to predict particle agglomeration and deagglomeration with resultant emitted aerodynamic particle size distributions (APSDs);
calibrating, by the one or more processor, the CFD-DEM virtual whole-lung model;
validating, by the one or more processor, the CFD-DEM virtual whole-lung model; and,
determining, by the one or more processor, drug delivery efficiency and deposition patterns of a dry powder inhaler within the patient respiratory system using the CFD-DEM virtual whole-lung model.

15. The method of claim 14, further comprising determining, by the one or more processor, adhesion resulting from short-range surface force of agglomeration in the patient respiratory system using the CFD-DEM virtual whole-lung model.

16. The method of claim 14, further comprising determining, by the one or more processor, carrier-API interactions in dry powder inhalers using the CFD-DEM virtual whole-lung model.

17. The method of claim 14, further comprising determining, by the one or more processor, effect of lactose carrier shape on drug delivery efficiency using the CFD-DEM virtual whole-lung model.

18. The method of claim 14, further comprising determining, by the one or more processor, effect of dry powder inhaler flow channel design on drug delivery efficiency using the CFD-DEM virtual whole-lung model.

19. The method of claim 14, further comprising determining, by the one or more processor, drug delivery deposition patterns within the patient respiratory system using the CFD-DEM virtual whole-lung model.

20. The method of claim 14, wherein the CFD-DEM virtual whole-lung model includes a pulmonary route from mouth and nose to alveoli, and the step of generating the CFD-DEM virtual whole-lung model is further defined as generating, by the one or more processor, the CFD-DEM virtual whole-lung model including the pulmonary route from mouth and nose to alveoli.
